(19) United States
(12) Patent Application Publication
(10) Pub. No.: US 2023/0321126 A1
(43) Pub. Date: Oct. 12, 2023

(54) Micronutrient combination to inhibit coronavirus cell infection

(71) Applicant: MATTHIAS W RATH, HENDERSON, NV (US)

(72) Inventors: Aleksandra Niedzwiecki, Henderson, NV (US); MATTHIAS W Rath, (DE); Vadim Ivanov, Castro Valley, CA (US); Anna Goc, Sanjose, CA (US)

(73) Assignee: MATTHIAS W RATH, HENDERSON, NV (US)

(21) Appl. No.: 18/021,201

(22) PCT Filed: Aug. 13, 2021
(86) PCT No.: PCT/IB2021/057477

§ 371 Date: Feb. 14, 2023

Related U.S. Application Data
(60) Provisional application No. 63/065,564, filed on Aug. 14, 2020.

(51) Int. Cl. A61K 31/7048; A61K 31/05; A61K 31/12; A61K 36/82; A61K 36/31; A61K 31/375; A61K 31/708; A61K 45/06; A61P 31/14

(52) U.S. Cl. CPC A61K 31/7048; A61K 31/05; A61K 31/12; A61K 36/82; A61P 31/14; A61K 31/375; A61K 31/708; A61K 45/06; A61K 36/31

(57) ABSTRACT

The way the SARS-CoV-2 virus infects the cell is a complex process and comprises four main stages: attachment to the cognate receptor, cellular entry, replication and cellular egress. Targeting binding of the virus to the host receptor in order to prevent its entry has been of particular interest. We tested 56 polyphenols, including plant extracts, brazilin, theaflavin-3,3′-digallate, and curcumin displayed the highest binding with the receptor-binding domain of spike protein, inhibiting viral attachment to the human angiotensin-converting enzyme 2 receptor, and thus cellular entry of pseudo-typed SARS-CoV-2 virions. Both, theaflavin-3,3′-digallate at 25 μg/ml and curcumin above 10 μg/ml concentration, showed binding with the angiotensin-converting enzyme 2 receptor reducing at the same time its activity in both cell-free and cell-based assays. Our study also demonstrates that brazilin and theaflavin-3, 3′-digallate, curcumin, decrease the activity of transmembrane serine protease 2 both in cell-free and cell-based assays and moderately increased endosomal/lysosomal pH.

### CROSS REFERENCE TO RELATED APPLICATIONS

This application claims priority to PCT/IB2021/057477 filed on Aug. 13, 2021. The disclosure of PCT application PCT/IB2021/057477 is hereby incorporated by this reference in their entirety for all of their teachings.

### FIELD OF STUDY

This application discloses micronutrient composition to mitigate SARS-CoV-2 virus cell infection at the cellular level by inhibiting cellular entry, cell surface attachment and egress of the virus in mammalian cell.

### BACKGROUND

The emergence and rapid spread of COVID-19 resulting in severe respiratory problems and pneumonia is destroying global health and economy. To date (https://covid19.who.int/ Jul. 30, 2020), COVID-19 has affected over 16.8 million people and caused more than 662,000 deaths worldwide. Sequencing the whole genome of a virus from patient samples (Zhu et al., 2020) identified a new coronavirus which was named severe acute respiratory syndrome coronavirus-2 (SARS-CoV-2) by the Coronavirus Study Group (CSG) of the International Committee on Taxonomy of Viruses (Gorbalenya et al., 2020). The disease caused by the virus was named coronavirus disease 2019 (COVID-19) by the World Health Organization (WHO). Since the genome of the novel SARS-CoV-2 has been identified (Zhu et al., 2020) the understanding how SARS-CoV-2 enters human cells is a high priority for deciphering its mystery and curbing its spread.

The cell entry mechanism of SAPS-CoV has been extensively studied. To enter host cells, corona viruses first bind to a cell surface receptor for viral attachment, subsequently enter cell endosomes, and eventually fuse viral and lysosomal membranes (F. Li, (2016). Coronavirus entry is mediated by a spike protein anchored on the surface-of the virus. On mature viruses, the spike protein is present as a trimer, with three receptor-binding S1 heads sitting on top of a trimeric membrane fusion S2 stalk.

The spike S1 protein on SARS-CoV-2 contains a receptor-binding domain (RBD) that specifically recognizes its cellular receptor-angiotensin-converting enzyme 2 (ACE2). As such, the receptor-binding domain (RBD) on SARS-CoV-2 spike protein part S1 head binds to a target cell using, human ACE2 (hACE2) receptor on the cell surface and is proteolytically activated by human proteases. Coronavirus entry into host cells is an important determinant of viral infectivity and pathogenesis, it is also a major target for various therapeutic intervention strategies (L. Du et al, 2009, L. Du et al 2017). Since the RBD of SARS-CoVs and other pathogenic human coronaviruses is also a common target of human antibodies this domain is a promising candidate for use in antibody-based diagnostic assays.

Cellular receptor for the virus binding is angiotensin-converting enzyme II or ACE2 which is an integral membrane protein present on many cells throughout the human body with its strong expression in the heart, vascular system, gastrointestinal system, and kidneys as well as in type H alveolar cells in the lungs. This protein has attracted much attention as the entry point for coronaviruses, including SARS-CoV-2 to hook into and infect a wide range of human cells (Zhu et al 2019) (li 2003, Hoffman 2005).

Since several mechanisms are involved in the pathogenicity of Cod′ the most effective approach to their control is by using natural compounds which by their nature are able to affect simultaneously multiple biochemical processes in cellular metabolism. We need a multipronged solution to prevent and mitigate the entry and egress of the virus from mammalian cell.

### SUMMARY

The instant micronutrient composition inhibits, treats, impairs attachment, penetration, multiplications, maturation and release of a coronavirus SARS-Cov-2 virus in a mammalian cell. In one embodiment, micronutrient composition comprises of phytochemical, phenolic acid, plant extracts, flavonoid, stilbenes, alkaloids, terpene, vitamin, volatile oil, mineral, fatty acids polyunsaturated and fatty acids monounsaturated. In one embodiment, the micronutrient composition comprising of phytochemicals in combination with other vitamins prevents various steps of infection in a mammal. In another embodiment, the micronutrient, composition comprising of phytochemicals, polyphenols, plant extracts, volatile oils, fatty acids polyunsaturated, fatty acids monounsaturated; and lipid soluble vitamins are used for inhibiting and treating Covid-19 infection and disease. In one embodiment, the micronutrient composition comprising of plant extracts as micronutrient combination block ACE 2 receptor expression and SARS CoV-2 spike domain-receptor binding domain site (RBD).

In another embodiment, the micronutrient composition deactivates attachment to cognate receptor, cellular entry and enhances cellular egress of the SARS CoV-2 virus. In another embodiment, the micronutrient composition comprising of phenolic acids such as curcumin, flavonoids such as luteolin, baicalein, hesperidin, brazilin individually and in combination with other micronutrient stops viral RBI) binding a receptor, hence help treat the mammal after the infection has occurred.

In one embodiment, a micronutrient composition to treat a SARS-CoV-2 virus infection by inhibiting attachment to a cognate receptor, cellular entry, replication and cellular egress of the SARS-CoV-2 virus in a mammal comprises of a phenolic acid, plant extracts, flavonoid, stilbenes, alkaloid, terpene, vitamin, volatile oil, mineral, fatty acids polyunsaturated, fatty acids monounsaturated individually or in combination thereof, wherein the phenolic acid are at least one of a tannic acid, (+) epigallocatechin gallate, (−)-gallo catechin gallate, curcumin and a combination thereof, wherein plant extracts are at least one of a quercetin, cruciferous extract, turmeric root extract, green tea extract or black tea extract and resveratrol, wherein flavonoid is at least one of a hesperidin, brazilin, phloroglucinol and myricetin, wherein alkaloid is at least one of a palmatine and usnic acid, wherein terpene is at least one of a D-limonene and carnosic acid, wherein stilbenes is a trans-resveratrol, wherein the vitamin is at least one of a vitamin C, vitamin E, vitamin B1, vitamin B2, vitamin B3, vitamin B6, vitamin B12, folate, biotin and a combination thereof, wherein the volatile oils are at least one of a eugenol oil from clove oil, oregano oil, carvacrol, cinnamon oil, thyme oil, trans-trans-cinnamaldehyde, wherein fatty acid polyunsaturated are at least one of a linolenic acid, eicosapentaenoic acid, docosahexaenoic acid, linoleic acid, wherein fatty acid monounsaturated are at least one of a oleic acid and petroselinic acid.

The micronutrient composition in one embodiment comprises of micronutrients as a composition are present in between a range of: the tannic acid 1 mg-200 mg, (+) epigallocatechin gallate 1 mg-5000 mg, (−)-gallo catechin gallate 1 mg-5000 mg, curcumin 1 mg-10000 mg, quercetin 1 mg-2000 mg, cruciferous extract 1 mg-5000 mg, turmeric root extract 1 mg-30000 mg, green tea extract 1 mg-20000 mg, resveratrol 1 mg-50000 mg, hesperidin 1 mg-2000 mg, brazilin 1 mg-1000 mg, phloroglucinol 1 mg-100 mg, myricetin 1 mg-1000 mg, wherein alkaloid is at least one of a palmatine and usnic acid, D-limonene 1 mg-1,500 mg, carnosic acid 1 mg-700 mg, trans-resveratrol 1 mg-3,000 mg, vitamin C 10 mg-100000 mg, vitamin E 1 mg-3,000 mg, vitamin B1 1 mg-3000 mg, vitamin B2 1 mg-2000 mg, vitamin B3 1 mg-3000 mg, vitamin B6 1 mg-3000 mg, vitamin B12 10 mcg-2000 mcg, folate 1 mcg-3000 mcg, biotin 1 mg-20000 mg, eugenol oil from clove oil 1 mg-300 mg, oregano oil 1 mg-1000 mg, carvacrol 1 mg-500 mg, cinnamon oil 1 mg-1000 mg, thyme oil 0.1 mg-100 mg, trans-trans-cinnamaldehyde 1 mg-4000 mg, linolenic acid 1 mg-8000 mg, eicosapentaenoic acid 1 mg-8000 mg, docosahexaenoic acid 1 mg-8000 mg, linoleic acid 1 mg-8000 mg, oleic acid 1 mg-20000 mg and petroselinic acid 1 mg-4000 mg.

The other embodiments are combinations of subsets of the micronutrient compositions for different functions for a multifaceted inhibition of viral infection and in narrower range. For example the micronutrient composition consisting of quercetin 1 mg-2000 mg, cruciferous extract 1 mg-5000 mg, turmeric root extract 1 mg-30000 mg, green tea extract 1 mg-20000 mg, resveratrol 1 mg-50000 mg and a combination thereof to inhibit attachment to a cognate receptor for cellular entry of a SARS-CoV-2 virus in a mammal. In another embodiment, the micronutrient composition consists of the tannic acid 1 mg-200 mg, (+) epigallocatechin gallate 1 mg-5000 mg, (−)-gallo catechin gallate 1 mg-5000 mg, curcumin 1 mg-10000 mg, hesperidin 1 mg-2000 mg and brazilin 1 mg-1000 mg to decrease the activity of a protease enzyme and inhibit the replication and cellular egress of the SARS-CoV-2 virus in the mammal.

In yet another embodiment the micronutrient composition consists of the tannic acid 10 mg-100 mg, (+) epigallocatechin gallate 10 mg-4000 mg, (−)-gallo catechin gallate 10 mg-4000 mg, curcumin 10 mg-8000 mg, hesperidin 10 mg-1000 mg and brazilin 10 mg-800 mg to decrease the activity of a protease enzyme and inhibit the replication and cellular egress of the SARS-CoV-2 virus in the mammal.

A micronutrient composition for the prevention and treatment of viral infections that use cellular receptors for viral entry on the surface of epithelial cells, endothelial cells and/or other cell types is disclosed. A micronutrient composition for the prevention and treatment of viral infections/diseases that use angiotensin converting enzyme 2 (ACE2) receptors on the surface of epithelial cells, endothelial cells and other cell types for viral entry is disclosed.

A micronutrient composition for the prevention and treatment of infections with Severe acute respiratory syndrome-related coronaviruses (SARS-CoV-1) that uses angiotensin converting enzyme 2 (ACE2) receptors on the surface of epithelial cells, endothelial cells and other cell types for viral entry is disclosed.

A micronutrient composition for treating, inhibiting of infections with SARS-CoV-1 that uses angiotensin converting enzyme 2 (ACE2) receptors on the surface of epithelial cells, endothelial cells and other cell types for viral entry, binding to RBD to inhibit viral spike attachment, inhibiting cellular proteases that are involved in transmembrane activity that facilitate the binding and endosomal egress of SARS-CoV-2, moderately increasing cellular pH are disclosed.

A micronutrient composition for the treating and inhibiting of infections with severe acute respiratory syndrome coronavirus 2 (SARS-CoV-2/COVID-19) that uses angiotensin converting enzyme 2 (ACE2) receptors on the surface of epithelial cells, endothelial cells and other cell types for viral entry is disclosed. A micronutrient composition for oral intake at physiological concentration is disclosed.

A micronutrient composition for intravenous application, for use as aerosol, inhalation solution, nasal or mouth spray, toothpaste, mouthwash, skin cream, skin patch, suppository or any other medically acceptable form of application is disclosed. A micronutrient composition where the compounds are applied in form of a physical mixture of the individual components is disclosed.

A micronutrient composition where two or more of the compounds are chemically bound/covalently linked to each other. A micronutrient composition comprising carriers, stabilizers and/or other medically acceptable additives is disclosed. In one embodiment, combination of polyphenols and plant extract (PB-) were tested. In one embodiment, formula 1, formula 2, formula 3 and formula 4 were also tested to mitigate viral infection.

A micronutrient composition where one or more of the compounds are covalently linked to a carrier molecule. A micronutrient composition to be applied to the patient in form of nanoparticles or any other medically acceptable delivery form is disclosed.

### BRIEF DESCRIPTION OF DRAWINGS

Example embodiments are illustrated by way of example and not limitation in the figures of the accompanying drawings, in which like references indicate similar elements and in which:

FIG. 1 shows effects of a combination of micronutrients containing active plant compounds and extracts (Formula 1) on ACE2 expression in small lung alveolar cells.

FIG. 2 shows the effects of Formula 1 on blocking SARS CoV-2 spike domain—RBD site.

FIG. 3 shows the effects of Formula 1 without one of its components (cruciferex) effects on viral RBD binding to ACE2 receptor.

FIG. 4 shows that Formula 1 effects on viral RBD binding is not affected when one of its components (cruciferex) is replaced by broccoli extract.

FIG. 5 shows the effects of other micronutrient compositions (Formula 2, 3 and 4) on blocking of the viral RBD binding to ACE2 receptor.

FIG. 6 shows different micronutrient combinations with Formula 1 affect inhibition of RBD binding to ACE2 receptor.

FIG. 7 shows enhanced RBD binding inhibition of Formula 1 by its combination with Baicalin, Luteolin, and Hesperidin.

FIG. 8 shows enhancement of RBD binding inhibition of Formula 1 w/o cruciferex by its combination with Theaflavin 3′3 digallate.

FIG. 9 shows dose-dependent binding of RBD-SARS-CoV-2 to immobilized hACE2 receptor.

FIG. 10 shows dose-dependent binding of A546 cells expressing SARS-CoV-2 eGFP-spike protein, in the presence of indicated polyphenols at different concentrations, to soluble hACE2 receptor.

FIG. 11A, FIG. 11B, and FIG. 11C shows viability of A549 cells after using TF-3, curcumin, and Brazilin.

FIG. 12A, FIG. 12B, and FIG. 12C shows dose-dependent binding of SARS-CoV-2 spike protein-encapsulated pseudo-virions to A549 cells stably overexpressing human ACE2 receptor evaluated after 1 h incubation.

FIG. 13A, FIG. 13B, and FIG. 13C shows dose-dependent binding of SARS-CoV-2 spike protein-encapsulated pseudo-virions to A549 cells stably overexpressing hACE2 receptor evaluated after 3 h incubation.

FIG. 14A, FIG. 14B, and FIG. 14C shows SARS-CoV-2 eGFP-luciferase-pseudo virion cellular entry for Attachment and entry of SARS-CoV-2 pseudo-virion with encapsulated eGFP-luciferase spike protein was evaluated without spinfection after 48 h incubation.

FIG. 15A, FIG. 15B, and FIG. 15C shows SARS-CoV-2 eGFP-luciferase-pseudo-virion cellular entry Attachment and entry of SARS-CoV-2 pseudo-virion with encapsulated eGFP-luciferase spike protein was evaluated with spinfection after 48 h incubation.

FIG. 16A, FIG. 16B, and FIGS. 16C, 16D, FIG. 16E, and FIGS. 16F, 16G, FIG. 16H, and FIG. 16I, FIG. 16J and FIG. 16H shows effect of selected polyphenols on fusion to human ACE2 receptor overexpressing A549 cells expressing ACE-2 receptor.

FIG. 17 shows Effect of selected polyphenols on fusion to human ACE2 receptor overexpressing A549 cells Quantitative analysis of formed syncytia.

FIG. 18A, FIG. 18B, and FIG. 18C shows Effects of selected polyphenols on cellular membrane associated proteases. (A) Binding of indicated polyphenols at different concentrations to hACE2 receptor.

FIG. 19A Activity of recombinant hACE2 upon treatment with indicated polyphenols at different concentrations and FIG. 19B shows Activity of cellular hACE2 upon treatment with indicated polyphenols at different concentrations.

FIG. 20 shows western blot analysis of hACE2 and TMPRSS2 expression in A549 cells upon treatment with indicated polyphenols with different concentration for 48 h period.

FIG. 21A, FIG. 21B and FIG. 21C shows effects of selected polyphenols on cellular membrane associated proteases.

FIG. 22A and FIG. 22B shows activity of recombinant TMPTSS2 upon treatment with indicated polyphenols at different concentrations.

FIG. 23 shows western blot analysis of hACE2 and TMPRSS2 expression in A549 cells upon treatment with indicated polyphenols with different concentration for 48 h period.

FIG. 24A effect of selected polyphenols on cathepsin L activity of purified cathepsin L enzyme upon treatment with indicated polyphenols at different concentrations and FIG. 24B shows activity of cellular cathepsin L upon treatment with indicated polyphenols at different concentrations.

FIG. 25 Western blot analysis of Cathepsin-L expression in A549 cells treated with indicated polyphenols with different concentration for 24 h.

FIG. 26 shows and quantified as band densitometry analysis indicating changes in protein expression.

FIG. 27A, FIG. 27B, FIG. 27C and FIG. 27D shows intracellular/lysosomal pH measurement. pHrodo™ Green AM dye and additional incubation for 30 min. at 37° C.

FIGS. 28A, 28B, 28C, 28D, 28E, 28F, 28G and 28H shows endosomal pH measurement in A549 cells treated with indicated polyphenols at different concentrations for 3 h at 37° C.

FIG. 29A, FIG. 29B and FIG. 29C shows effect of combination of polyphenols and plant extract (PB) on receptor binding.

FIGS. 30 and 30B shows effects of BP on the attachment and entry of pseudo-virions encapsulated with eGFP-luciferase spike protein.

FIG. 31A, FIG. 31B and FIG. 31C effect of PB on host cellular receptors and proteases.

FIG. 32A, FIG. 32B and FIG. 32C shows enzyme activity due to PB.

FIG. 33A, FIG. 3313, and FIG. 33C shows activity of Cathepsin with PB treatment.

FIG. 34A and FIG. 34B shows effects of PB on furin activity.

FIG. 35 shows effect of PB on viral RNA polymerase.

Others features of the present embodiments will be apparent from the accompanying drawings and from the detailed description that follows.

### DETAILED DESCRIPTION

The life cycle of the virus with the host consists of the following 5 steps: attachment, penetration, biosynthesis, maturation and release. Once viruses bind to host receptors (attachment), they enter host cells through endocytosis or membrane fusion (penetration). Once viral contents are released inside the host cells, viral RNA enters the nucleus for replication. Viral mRNA is used to make viral proteins (biosynthesis). Then, new viral particles are made (maturation) and released. Coronaviruses consist of four structural proteins; Spike (S), membrane (M), envelope (E) and nucleocapsid (N). Spike is composed of a transmembrane trimetric glycoprotein protruding from the viral surface, which determines the diversity of coronaviruses and host tropism. Our earlier study showed that a natural micronutrient composition containing vitamin C, minerals, amino acids and plant extracts was effective in significantly decreasing cellular ACE2 expression in human lung alveolar epithelial and vascular endothelial cells. These inhibitory effects persisted under pro-inflammatory conditions associated with infections.

Here, we present experimental results showing a potential of representative polyphenols to inhibit the binding and entry of SARS-CoV-2 virions. Using standard and recently developed methodology, we report that, among 56 tested phenolic compounds, including plant extracts, brazilin, TF-3, and curcumin have the highest binding affinity to the viral RBD of SARS-CoV-2 spike protein. Moreover, concurrent experiment with SARS-CoV-2 pseudo-viral particles revealed that these three polyphenols have the pronounced inhibitory effect on viral binding and cellular entry. We also discovered that TF-3 and curcumin inhibit the activity of TMPRSS2 and cathepsin L proteases that facilitate the binding and endosomal egress of SARS-CoV-2, and modestly increase lysosomal pH, as does brazilin. In conclusion, this study documents anti-SARS-CoV-2 activity of these three polyphenols, providing a scientific basis for their further investigations in in vivo and clinical studies.

In this study we tested the efficacy of different nutrient compositions containing vitamins, minerals, polyphenols and plant components on key aspects of CoV infectivity: cellular ACE2 expression and interference with viral RBD binding to ACE2 receptors. We also tested the effects of individual natural compounds (polyphenols, fatty acids, volatile oils and others) on RED binding inhibition to ACE2 receptor. The compounds can be used individually or in combination.

The results show that all micronutrient compositions were effective in lowering RBD binding to ACE2 receptor, however a specific composition of plant-derived compounds (Formula 1) was more effective that other in decreasing CoV infectivity at the cellular level (92% inhibition of ACE2 expression) and 97% inhibition of viral RBD binding and it should be considered as safe and affordable approach in controlling current COVID-19 pandemic.

### Material and Methods

Cell cultures: Human Small Airways Epithelial Cells (SAEC, purchased from ATCC) were cultured in Airways Epithelial Cells growth medium (ATCC) in plastic flasks at 37° C. and 5% CO₂. For the experiment SAEC, passage 5-7, were plated to collagen-covered 96 well plastic plates (Corning) in 100 μL growth medium and were grown to confluent layer for 4-7 days.

Micronutrient composition: The micronutrient combination used in our experiments developed at the Dr. Rath Research Institute (San Jose, Ca). The composition of all 4 formulas tested is presented in Table 1.

| | | Formula 2 | | Formula 3 | | Formula 4 | Physiological |
|---|---|---|---|---|---|---|---|
| Micronutrient | Formula 1 | (epiQ) | | (healthy imm) | | (biolymix) | dose range |
| Vitamin C | | 710 | mg | 400 | mg | | 10 mg-100,000 mg |
| Vitamin E | | | | 30 | mg | | 1 mg-3,000 mg |
| Vitamin B1 | | | | 2.4 | mg | | 1 mg-3,000 mg |
| Vitamin B2 | | | | 2.6 | mg | | 1 mg-2,000 mg |
| Vitamin B3 | | | | 16 | mg | | 1 mg-3,000 mg |
| Vitamin B6 | | | | 3.4 | mg | | 1 mg-1000 mg |
| Vitamin B12 | | | | 5 | mcg | | 10 mcg-2,000 mcg |
| Folate | | | | 400 | mcg | | 1 mcg-3,000 mcg |
| Biotin | | | | 60 | mcg | | 1 mg-20,000 mg |
| Pantothenic acid | | | | 10 | mg | | 1 mg-20,000 mg |
| Zinc | | | | 10 | mg | | 1 mg-1,000 mg |
| Selenium | | 30 | mcg | 10 | mcg | | 2 mcg-500 mcg |
| Copper | | 2 | mg | | | | 0.01 mg-20 mg |
| Manganese | | 1 | mg | | | | 1 mg-30 mg |
| Iodine (kelp) | | | | | | 302 mcg | 0.01 mg-2 mg |
| L-Lysine | | 1000 | mg | | | | 1 mg-40,000 mg |
| L-arginine | | 500 | mg | | | | 1 mg-30,000 mg |
| L-proline | | 750 | mg | | | | 1 mg-20,000 mg |
| N-acetylcysteine | | 200 | mg | | | | 1 mg-30,000 mg |
| Alpha lipoic acid | | | | 40 | mg | | 1 mg-5,000 mg |
| Luteolin | | | | | | 75 mg | 0.1 mg-100 mg |
| Quercetin | 400 mg | 50 | mg | | | | 1 mg-2,000 mg |
| Cruciferous extr. | 400 mg | | | | | | 1 mg-5,000 mg |
| Turmeric root extr. | 300 mg | | | | | | 1 mg-30,000 mg |
| Green tea extr | 300 mg | 1000 | mg | | | | 1 mg-20,000 mg or |
| (EGCG) | | | | | | | (1 mg-5,000 mg |
| | | | | | | | as EGCG) |
| Resveratrol | 50 mg | | | | | | 1 mg-50,000 mg |
| Ginger root | | | | 200 | mg | | 1 mg-20,000 mg |
| Aronia berry extr | | | | 200 | mg | | 1 mg-20,000 mg |
| Lychee fruit extr | | | | 200 | mg | | 1 mg-30,000 mg |
| Tart cherry fruit extr | | | | 200 | mg | | 1 mg-30,000 mg |
| Fucoidan | | | | 60 | mg | | 1 mg-15,000 mg |
| White mulberry extr | | | | 50 | mg | | 1 mg-20,000 mg |
| Medium chain | | | | | | 800 mg | 1 mg-70,000 mg |
| triglycerides | | | | | | | |
| Skullcap root extr | | | | | | 600 mg | 1 mg-5,000 mg |
| Rosemary leaf extr | | | | | | 450 mg | 1 mg-10,000 mg |
| Royal Jelly | | | | | | 500 mg | 1 mg-10,000 mg |

In addition we tested the effects of individual compounds; polyphenols, fatty acids, volatile oils and other compounds as presented in Table 2:

| Class of compounds | Compound | Physiological dose range |
|---|---|---|
| Polyphenols | Tannic acid | 1 mg-200 mg |
| (0.1 mg/ml) | Curcumin | 1 mg-10,000 mg |
| Flavonoids | Hesperidin | 1 mg-2,000 mg |
| | (+) Epigallocatechin gallate (EGCG) | 1 mg-5,000 mg |
| | (−)-gallocatechin gallate | 1 mg-5,000 mg |
| | Brazilin | 1 mg-1,000 mg |
| Plant extracts | Tea extract (85% catechins) | 0.1 mg-10,000 mg |
| (0.1 mg/ml) | Tea extract (85% theaflavins) | 0.1 mg-10,000 mg |
| | Theaflavin 3′3 di-gallate | 1 mg-30,000 mg |
| Volatile oils (5%) | Clove oil | 1 mg-400 mg |
| | Eugenol from clove oil | 1 mg-300 mg |
| | Oregano oil | 1 mg-1,000 mg |
| | Carvacrol (from oregano oil) | 1 mg-500 mg |
| | Cinnamon oil | 1 mg-1,000 mg |
| | Trans-trans-cinnamaldehyde | 1 mg-4,000 mg |
| | Thyme oil | 0.1 mg-100 mg |
| Fatty acids | linolenic acid, eicosapentaenoic acid, | 1 mg-8,000 mg |
| Polyunsaturated: | docosahexaenoic acid, linoleic acid | |
| Fatty acids | oleic acid | 1 mg-20,000 mg |
| Monounsaturated; | Petroselinic acid | 1 mg-4,000 mg |
| Lipid soluble vitamins | Vitamin A (retinol) | 10 IU-50,000 IU |

Cell supplementation: The micronutrient mixture was dissolved in 0.1N HCl according to US Pharmacopeia protocol (USP 2040) and designated as a stock solution. For ACE2 expression experiments SAEC cells were supplemented with indicated doses of the formulation in 100 μL/well cell growth medium for 3-7 day. Applied nutrient concentrations were expressed as millionth parts of a stock concentration per ml (mpsc/mL).

ACE-2 ELISA assay: Culture plate wells were washed twice with phosphate buffered saline (PBS) and fixed with 3% formaldehyde/0.5% Triton X100/PBS solution for 1 h at 4° C., then washed four times with PBS. 200 μL of 1% bovine serum albumin BSA, Sigma) in PBS was added and plate was incubated at 4° C. overnight. Rabbit polyclonal anti ACE-2 antibodies (Sigma) were added to 100 μL 1% BSA/PBS for 1.5 h incubation at room temperature (RT). After three wash cycles with 0.1% BSA/PBS wells were supplied with 100 μL anti-rabbit IgG antibodies conjugated with horse radish peroxidase (HRP, Sigma) for 1 h at RT. After three wash cycles with 0.1% BSA/PBS the HRP activity retained was determined by incubation with 100 μL TMB substrate solution (Sigma) for 20 min at RT, followed by the addition of 50 μL of 1N H2504 and optical density measurement at 450 nm with micro plate reader (Molecular Devices). Results are expressed as a percentage of experimental addition-free control (mean+/−SD, n=6). Non-specific control (wells incubated without anti ACE2 antibodies) mean value (n=6) was subtracted from all sample values.

RBD binding: This assay was performed using GenScript SARS-CoV-2 surrogate virus neutralization test kit that can detect either antibody or inhibitors that block the interaction between the receptor binding domain (RBD) of the viral spike protein with ACE2 cell surface receptor. All test sample with indicated concentrations, and positive and negative controls (provided by the manufacturer) were diluted with the sample dilution buffer with a volume ratio of 1:9. In separate tubes, HRP conjugated RBD was also diluted with the HRP dilution buffer with a volume ratio of 1:99. Biding/neutralization reaction was performed according to manufacturer's protocol. Briefly, diluted positive and negative controls as well as the test samples with indicated concentrations were mixed with the diluted HRP-RBD solution with a volume ratio of 1:1 and incubated for 30 minutes in 37° C. Next, 100 μl each of the positive control mixture, negative control mixture, and the test sample mixtures were added to the corresponding wells with immobilized ACE2 receptor and incubated for 15 minutes at 37° C. Subsequently, the plates were washed four times with 260 μl/well of the 1× wash solution and TMB solution was added to each well (100 μl/well). Plates were incubated in the dark at room temperature for up to 5 minutes. Next, 50 μl/well of stop solution was added to quench the reaction and the absorbance was measured immediately in plate reader at 450 nm. Experiment was performed three times in duplicates. Data are presented as % of control.

Cell lines and pseudo-viruses: Human alveolar epithelial cell line A549 was obtained from ATCC (American Type Culture Collection) (Manassas, VA). Human alveolar epithelial cell line A549, stably overexpressing hACE2 receptor (hACE2/A549), and eGFP-luciferase-SARS-CoV-2 spike glycoprotein pseudo-typed particles were obtained from GenScript (Piscataway, NJ). Cell lines were cultured in Dulbecco's Modified Eagle's Medium (DMEM) supplemented with 10% fetal bovine serum (FBS), 100 U/ml penicillin, and 100 μg/ml streptomycin. Pseudo-typed ΔG-luciferase (G [symbol] ΔG-luciferase) rVSV was purchased from Kerafast (Boston, MA). Bald pseudo-virus particles with eGFP and luciferase (eGFP-luciferase-SARS-CoV-2 pseudo-typed particles) were purchased from BPS Bioscience (San Diego, CA). Lentiviral particles encoding human TMPRSS2 were from Addgene (Watertown, MA). All antibodies were from R&D Systems (Minneapolis, MN) if not specified otherwise.

Test compounds, antibodies, recombinant proteins and inhibitors: Curcumin, tea extract standardized to 85% theaflavins, theaflavin-3,3′-digallate, gallic acid, tannic acid, Andrographis paniculata extract, andrographolide, licorice extract, glycyrrhizic acid, broccoli extract, L-sulforaphane, usnic acid, malic acid, D-limonene, and ammonia chloride with purity between 95-99%, according to the manufacturer, were purchased from Sigma (St. Louis, MO). All other polyphenols and camostat mesylate, with purity between 95-99% according to the manufacturer, were obtained from Cayman Chemical Company (Ann Arbor, MI). For screening study, test compounds were prepared as 10 mg/ml (25% DMSO) working stock solution and for the rest of experiments as 1.0 mg/ml (1% DMSO) and 10 mg/ml (10% DMSO). All antibodies were from Santa Cruz Biotechnology (Santa Cruz, CA). TMPRSS2 recombinant protein was from Creative BioMart (Shirley, NY).

Receptor binding and entry assays: SARS-CoV-2 RBD binding to hACE2. Binding reaction was performed using a SARS-CoV-2 Surrogate Virus Neutralization Test Kit that can detect either antibodies or inhibitors that block the interaction between the RBD-SARS-CoV-2 spike protein with the hACE2 receptor (GenScript, Piscataway, NJ). For screening, phenolic compounds or plant extracts (at 100 μg/ml concentration) were incubated with HRP-conjugated RBD-SARS-CoV-2 spike 51 domain for 30 min. at 37° C. Next, the samples that were incubated with RBD were transferred into a 96-well plate with immobilized hACE2 receptor and incubated for additional 15 min. at 37° C. Subsequently, the plates were washed four times with washing buffer and developed with TMB substrate solution for up to 5 min., followed by the addition of stop buffer. Optical density was measured immediately at 450 nm with a plate reader (Molecular Devices, San Jose, CA). Positive and negative controls were provided by the manufacturer. Control was 0.25% DMSO. Results are expressed as a percentage of polyphenol-free control (mean+/−SD, n=6).

SARS-CoV-2 pseudo-virus binding to hACE2. Binding reaction was performed using a GenScript-developed protocol with small applied adjustments. Briefly, eGFP-luciferase-SARS-CoV-2 spike 51 pseudo-virus was either pre-incubated at 37° C. with selected polyphenols (i.e., brazilin, TF-3, and curcumin) at concentrations ranging from 0-25 μg/ml for: 1) 1 h before adding into a plate with hACE2/A549 cells, 2) simultaneously added into the plate with hACE2/A549 cells, or 3) added into the plate with the hACE2/A549 cells 1 h post-treatment. A parallel experiment was performed; in which eGFP-luciferase-CoV-2 spike protein enveloped pseudo-virus was spin-inoculated at 1,200×g for 45 min. Samples were incubated for an additional 1 h, 3 h, and 48 h, at 37° C. After the incubation period, the plates were washed three times with washing buffer (provided by the manufacturer), and measured either HRP signal or luciferase activity using a Luciferase Glo Kit (Promega, Madison, WI). In 1 h and 3 h experiments, positive and negative controls were the same as those used in SARS-CoV-2 RBD binding to hACE2 assay, and were provided by the manufacturer. In 48 h experiments, the positive control was bald eGFP-luciferase-SARS-CoV-2 pseudo-typed particles, and the negative control was ΔG-luciferase rVSV pseudo-typed particles. Control was 0.025% DMSO. Results are expressed as a percentage of polyphenol-free control (mean+/−SD, n=6).

SARS-CoV-2 spike-protein-expressing cells binding to soluble hACE2. To transduce cells with eGFP-luciferase-SARS-CoV-2 spike 51 lentivirus vector (GenScript, Piscataway, NJ), A549 cells, seeded into a 6-well plate in the presence of complete growth medium, were treated with 8 μl/ml polybrene (Sigma, St. Louis, MO) for 30 min., followed by the addition of eGFP-luciferase-SARS-CoV-2 spike 51 lentivirus at MOI=40, and spin-inoculation at 1,000×g. for 1.5 h. After 24 h at 37° C. incubation, cells were fed with fresh complete growth medium. After 48 h post-inoculation, cells were detached with 1 mM EDTA, washed twice with 1×PBS (phosphate-buffered saline) supplemented with 3% FBS, and treated with indicated concentrations of polyphenols for 1 h, followed by incubation with 5 μg/ml of soluble hACE2 (Sigma, St. Louis, MO) for 1 h on ice. After washing three times with 3% FBS in 1×PBS, cells were transferred into plates with human monoclonal anti-ACE2 antibody at 10 μg/ml (Cayman Chemical Company, Ann Arbor, MI). After 1 h incubation, wells were washed three times with 3% FBS in 1×PBS, and fluorescence was measured at Ex/Em=488/535 nm wavelength with a plate reader (Tecan Group Ltd, Switzerland). Positive and negative controls were the same as those used in SARS-CoV-2 RBD binding to hACE2 assay, and were provided by the manufacturer. Control was 0.025% DMSO. Results are expressed as a percentage of polyphenol-free control (mean+/−SD, n=6).

Cell-cell fusion assay: Cell-cell fusion assay was performed according to Ou et al. [13]. Briefly, A549 cells transduced with eGFP-luciferase-SARS-CoV-2 spike 51 lentivirus vector (GenScript, Piscataway, NJ) were detached with 1 mM EDTA, treated with indicated concentrations of selected polyphenols, for 1 h at 37° C., and overlaid on 80-95% confluent human A549 lung epithelial cells overexpressing hACE2. After 4 h incubation at 37° C., images of syncytia were captured with a Zeiss AxioObserver A1 fluorescence microscope (Carl Zeiss Meditec, Dublin, CA). The positive control was 20 μg/ml anti-ACE2 antibody. Control was 0.025% DMSO. Results are expressed as a percentage of polyphenol-free control (mean+/−SD, n=3).

TMPRSS2 activity assay: Cellular TMPRSS2 activity assay was performed according to a previously published report. Briefly, hTMPRSS2/A549 cells were seeded in 48-well plates. 48 h or 3 h prior to the protease activity measurements, the cells were treated with selected polyphenols at 5.0-25 μg/ml concentrations. Next, cells were washed with DMEM without phenol red, and the protease activity was assessed by incubation of cells with the 200 μM fluorogenic substrate Mes-D-Arg-Pro-Arg-AMC in 50 mM PBS (pH=7.4) for 30 min. at 37° C. (Fisher Scientific, Pittsburgh, PA). Hydrolysis of the peptide was monitored by the measurement of fluorescence intensity, using a spectrofluorometer at Ex/Em=360/440 nm wavelength (Tecan Group Ltd, Switzerland). The positive control was 50 μM camostat mesylate. Control was 0.025% DMSO. Results are expressed as a percentage of polyphenol-free control (mean+/−SD, n=6).

Direct TMPRSS2 activity assay with recombinant enzyme was performed according to a previously published report [53]. To determine the inhibitory effect of selected polyphenols on the activity of isolated TMPRSS2 protein, 1 μM fluorogenic peptide Boc-Gln-Ala-Arg-AMC was added to the selected polyphenols diluted at 5.0-25 μg/ml concentrations. To this reaction 10 μM of TMPRSS2 enzyme in assay buffer (50 mM Tris pH=8, 150 mM NaCl) was added. Following 1 h incubation at RT, detection of fluorescent signal was performed using a spectrofluorometer at Ex/Em=360/440 nm wavelength (Tecan Group Ltd, Switzerland). The positive control was 100 μM camostat mesylate. Control was 0.025% DMSO. Results are expressed as a percentage of polyphenol-free control (mean+/−SD, n=6).

Cathepsin L activity assays: Cellular cathepsin L activity assays were performed utilizing a Cathepsin L Activity Assay Kit (Abcam, Cambridge, MA) according to the manufacturer's protocol. Briefly, A549 cells were seeded in 6-well plates and allowed to adhere for 24 h or until reaching 90-95% of confluence. Next, the cells were treated with indicated concentrations of selected polyphenols for an additional 24 h, washed with cold 1×PBS, and lysed using 100 μl of chilled CL buffer on ice for 5 min. The samples were then centrifuged for 2 min. at 4° C. to remove any insoluble material. Supernatants were collected and transferred to clean tubes that were kept on ice. Enzymatic reaction was set up by mixing treated sample wells containing 50 μl sample, 50 μl untreated sample (control), 50 μl background control, a positive control containing 5 μl reconstituted positive control in 45 μl CL buffer, and a negative control containing 5 μl reconstituted positive control in 45 μl CL buffer and 2 μl CL inhibitor. Next, 50 μl CL buffer and 1 μl 1 mM DTT were added to each well. Finally, 2 μl 10 mM CL substrate Ac-FR-AFC (200 μM final concentration) was added to each well, except background control samples. The plates were incubated at 37° C. for 1 h and fluorescence signal was measure at Ex/Em=400/505 nm wavelength with a spectrofluorometer (Tecan Group Ltd, Switzerland). Control was 0.025% DMSO. Results are expressed as a percentage of polyphenol-free control (mean+/−SD, n=6).

ACE2 activity assays: To determine the inhibitory effect of selected polyphenols on the activity of cellular hACE2 protein, hACE2/A549 cells were seeded in 48-well plates and allowed to adhere for 24 h or until reaching 99-100% of confluence. The cells were then treated with indicated concentrations of selected polyphenols for an additional 24 h, before being washed with cold 1×PBS, and enzymatic reaction was initiated by adding 200 μM fluorogenic substrate Mca-Y V AD AP K(Dnp)-OH. Finally, the plates were incubated at 37° C. for 1 h, and the fluorescence signal was measured at Ex/Em=320/405 nm wavelength with a spectrofluorometer (Tecan Group Ltd, Switzerland). Control was 0.025% DMSO. Results are expressed as a percentage of polyphenol-free control (mean+/−SD, n=6).

To determine the inhibitory effect of selected polyphenols on the activity of recombinant hACE2 protein, an ACE2 Activity Screening Assay Kit (BPS Bioscience, San Diego, CA) was used according to the manufacturer's protocol. Briefly, to ACE2 enzyme (0.2 mU/μl) the selected polyphenols at 5.0-25 μg/ml concentrations were added and the reaction mix was incubated for 15 min. at RT. The positive control was a sample containing only ACE2 enzyme, and the negative control was a sample containing ACE2 enzyme and 10% DMSO. ACE2 fluorogenic substrate (10 μM) was added to each well, and the plate was incubated for 1 h at RT. The fluorescence was measured at Ex/Em=535/595 nm wavelength using a spectrofluorometer (Tecan Group Ltd, Switzerland). Control was 0.025% DMSO. Results are expressed as a percentage of polyphenol-free control (mean+/−SD, n=6).

ACE2 binding assay: To determine the inhibitory effect of selected polyphenols on binding to the ACE2 receptor, an ACE2 Inhibitor Screening Assay Kit (BPS Bioscience, San Diego, CA) was used according to the manufacturer's protocol. Briefly, to ACE2 receptor immobilized on the plate (1.0 μg/ml), selected polyphenols at 5.0-25 μg/ml concentrations were added and the reaction mix was incubated for 1 h at RT. The positive control contained 20 μg/ml anti-ACE2 antibody in the sample, and the negative control was an addition-free sample. Next, the plate was washed three times with washing buffer, and SARS-CoV-2 spike protein at 1.0 μg/ml was applied for 1 h at RT, followed by washing three times, blocking with blocking buffer, and incubation with HRP-conjugated secondary antibody for an additional 1 h at RT. The plates were again washed three times with washing buffer, and chemiluminescence was measured using ECL substrate A and ECL substrate B mixed 1:1, using a micro-plate reader (Tecan Group Ltd, Switzerland). Control was 0.025% DMSO. Results are expressed as a percentage of polyphenol-free control (mean+/−SD, n=6).

Endosomal/lysosomal pH assay: Endosomal pH was assessed according to a previously reported protocol [54]. Briefly, A549 cells were seeded in 8-well chambers (MatTek, Ashland, MA), and, at 95-100% confluence, were treated with the indicated polyphenols at 5.0 and 25 μg/ml concentrations, followed by 3 h incubation at 37° C. in a 5% CO2. Acridine orange (Thermo Fisher Scientific, Waltham, MA) was added directly to each dish to reach a final concentration of 6.6 μg/ml. The cells were additionally incubated at 37° C. with 5% CO2 for 20 min. and washed three times with 1×PBS. Live Cell Imaging Solution (LCIS) (Thermo Fisher Scientific, Waltham, MA) was added to the wells, and images were taken using a Zeiss Axio Observer A1 fluorescence microscope with a 40× magnification. Control was 0.025% DMSO, whereas the positive control was 20 mM ammonia chloride. Results are expressed as a percentage of polyphenol-free control (mean+/−SD, n=3).

A concurrent experiment was performed using pHrodo™ Green AM Intracellular pH Indicator (Thermo Fisher Scientific, Waltham, MA) according to the manufacturer's protocol. Briefly, A549 cells were seeded at 95-100% confluence, treated with the indicated polyphenols at 5 and 25 μg/ml concentrations, and incubated for 24 h at 37° C. with 5% CO2. Next, 10 μl of the pHrodo™ Green AM dye was added to 100 μl of PowerLoad™ to facilitates uniform cellular loading of AM esters, and the whole dye solution was transferred into 10 ml of LCIS. The growth medium from cells was removed, cells were washed once with LCIS, and replaced with the pHrodo™ Green AM staining solution. The plate was incubated for 30 min. at 37° C., washed again with LCIS, and fluorescence was measured at Ex/Em=509/533 nm wavelength using a spectrofluorometer (Tecan Group Ltd, Switzerland). pH identification was performed based on standard curve, using an Intracellular pH Calibration Buffer Kit according to the manufacturer's protocol (Thermo Fisher Scientific, Waltham, MA). Briefly, after performing cellular experiment with pHrodo™ Green AM, cells were washed twice with LCIS, the LCIS was replaced with cellular pH calibration buffer at pH=4.5, supplemented with 10 μM of valinomycin and 10 μM of nigericin, and the cells were incubated at 37° C. for 5 min. Next, the fluorescence was measured Ex/Em=509/533 nm wavelength. These steps were repeated with the three additional cellular pH calibration buffers at pH=5.5, 6.5 and 7.5, respectively, to obtain altogether four data points that were plotted to get the pH standard curve. Control was 0.025% DMSO, whereas the positive control was 20 mM ammonia chloride. The experiment was repeated three times, each one in triplicates.

Viability assay: MTT assay was used to assess cell viability. Briefly, A549 cells were seeded into a 96-well plate at a cell density of 40,000 per well, and allowed to adhere for 24 h, followed by treatment with different concentrations of selected polyphenols for up to 48 h. Next, complete growth medium was replaced with a fresh one substituted with 5 mg/ml MTT, followed by incubation for 3 h at 37° C. After removing the culture medium, 100 μl of methanol was added and the absorbance was measured at 570 nm using a spectrophotometer (Molecular Devices, San Jose, CA). Control was 0.025% DMSO. Results are expressed as a percentage of polyphenol-free control (mean+/−SD, n=10).

Western blot analysis: A549 cells were treated with indicated concentrations of selected polyphenols and lysed using RIPA lysis buffer (Sigma, St. Louis, MO) supplemented with 1× Complete protease inhibitors (Roche Applied Science, Indianapolis, IN). The protein concentration was measured by the Dc protein assay (Bio-Rad, Hercules, CA). Proteins (50 μg/well) were separated on 8-16% gradient SDS-PAGE gels and transferred to a PVDF membrane. Specific proteins were detected with commercially available human anti-cathepsin L, anti-TMPRSS2, and anti-ACE2 monoclonal antibodies, all at 1:200 dilution, and anti-β-actin antibody as a loading control at 1:1000 dilution. Images were captured with Azure™ cSeries digital imaging system (Azure Biosystems, Dublin, CA) with auto-exposure settings. Densitometry was performed with NIH ImageJ software.

Plant-derived composition: The combination tested in this study consisted of 400 mg of quercetin, 400 mg of cruciferous plant extract, 300 mg of turmeric root extract, 300 mg of green tea extract (80% polyphenols) and 50 mg of resveratrol. A stock solution of this plant-derived combination was prepared in DMSO at 100 mg/ml and kept at −20° C. until analysis. For the experiments, the stock solution was diluted with 1×PBS (enzyme activity assays) or corresponding cell culture medium (cell expression assays) to final concentrations indicated in the figures.

Binding of SARS-CoV-2 pseudo-typed virions to hACE2 receptor: The experiment was executed according to GenScript recommendations with small modifications. Briefly, eGFP-luciferase-SARS-CoV-2 spike protein encapsulated pseudo-virions were incubated at 37° C. with 0-100 μg/ml of PB for 1 hour before it was either added into a monolayer of hACE2/A549 cells, simultaneously added to hACE2/A549 cells, or was added to the cells after 1 hour posttreatment. A parallel experiment was performed in which eGFP-luciferase-CoV-2 spike protein pseudo-virions were spin-inoculated at 1,250×g for 1.5 hours. Cells were incubated for an additional 1 hour, 3 hours and 48 hours, at 37° C. After the 1-hour and 3-hour incubation periods, cells were washed three times with washing buffer, and primary antibody against SARS-CoV-2 spike protein at 1:1000 dilution, followed by HRP-conjugated secondary antibody at 1:2500 dilution, were employed in ELISA assay. After the 48-hour incubation period (with or without spinfection), the transduction efficiency was quantified by recording of the luciferase activity, utilizing a luciferase assay system (Promega, Madison, WI) and a spectrofluorometer (Tecan Group Ltd., Switzerland). Positive and negative controls used in 1-hour and 3-hour experiments, were provided by the manufacturer. In the 48-hour experiments, the positive control was bald eGFP-luciferase-SARS-CoV-2 pseudo-typed particles, and the negative control was ΔG-luciferase rVSV pseudo-typed particles. Data are presented as a % of control without PB addition (mean+/−SD, n=6).

TMPRSS2 activity assay and its cellular expression: TMPRSS2 activity: TMPRSS2 activity assay in cell-based assay was performed according to previous report (22). Briefly, A549 cells overexpressing TMPRSS2 were treated with PB at 5.0 and 10 μg/ml concentrations 48 hours or 3 hours prior to the enzymatic activity assessment. Cells were then washed with growth medium (without added phenol red), and the activity was initiated by addition of the 200 μM fluorogenic substrate Mes-D-Arg-Pro-Arg-AMC for 30 minutes at 37° C. (Fisher Scientific, Pittsburgh, PA), using a spectrofluorometer at extension/emission=360/440 nm (Tecan Group Ltd., Switzerland). The positive control was 50 μM camostat mesylate. Data are presented as a % of control without PB addition (mean+/−SD, n=6).

Effect of PB on the activity of isolated TMPRSS2 protease, 1 μM fluorogenic peptide Boc-Gln-Ala-Arg-AMC was added to the PB diluted at 5.0 and 10 μg/ml concentrations followed by supplementation with 10 μM of TMPRSS2 (Creative BioMart, Shirley, NY) for 1 hour at RT. Fluorescence was assessed using a spectrofluorometer at extension/emission=360/440 nm (Tecan Group Ltd., Switzerland). The positive control was 100 μM camostat mesylate. Data are presented as a % of control without PB addition (mean+/−SD, n=6).

TMPRSS2 expression: Expression of TMPRSS2 in cells was performed using Human TMPRSS2 ELISA Kit (Novus Biologicals, Centennial, CO). Briefly, 48 hours prior to the analysis, A549 cells were treated with PB at 5.0 and 10 μg/ml concentrations. Next, all wells were washed with 1×PBS and lysed with CellLytic M buffer (MilliporeSigma, St. Louis, MO). Lysates were then processed according to procedure described in the ELISA manual provided by the manufacturer.

Cathepsin L activity assay and its cellular expression: Cathepsin L activity: Experiment was performed in cell lysates using a Cathepsin L Activity Assay Kit (Abcam, Cambridge, MA) according to the manufacturer's protocol. Briefly, 5×10⁶ A549 cells treated with PB at 5.0 and 10 μg/ml concentrations for 24 hours were washed with cold 1×PBS, and lysed 100 μl with CL buffer for 8 minutes. After 3 minutes of centrifuged for at 4° C., supernatants were collected and enzymatic reaction was set up by mixing 50 μl of treated sample, 50 μl of control sample, 50 μl of background control sample, 50 μl of positive and negative controls. 50 μl CL buffer and 1 μl 1 mM DTT was added next followed by addition of 2 μl of 10 mM CL substrate Ac-FR-AFC except for the background control. Samples were incubated at 37° C. for 1 hour, and fluorescence was recorded at extension/emission=400/505 nm with a fluorescence spectrometer (Tecan Group Ltd., Switzerland). Data are presented as a % of control without PB addition (mean+/−SD, n=6).

Effect of PB on the activity of isolated cathepsin L, a Cathepsin L Activity Screening Assay Kit (BPS Bioscience, San Diego, CA) was used according to the manufacturer's protocol. Briefly, PB at 5.0 and 10 μg/ml concentrations was added to cathepsin L (0.2 mU/μl) for 15 minutes at 22° C. prior to fluorogenic substrate (Ac-FR-AFC) (10 μM) addition and incubation for 60 minutes at RT. Positive control contained only cathepsin L, and negative control containing cathepsin L and cathepsin L inhibitor E64d (25 μM). The fluorescence was recorded at extension/emission=360/480 nm with a fluorescence spectrometer (Tecan Group Ltd., Switzerland). Data are presented as a percentage of control without PB addition (mean+/−SD, n=6).

Cathepsin L expression: Expression of cathepsin L in cells was performed using Western blot. Briefly, 48 hours prior to the analysis, A549 cells were treated with PB at 5.0, and 10 μg/ml concentrations. Next, cells were washed with 1×PBS, lysed, and processed according to procedure described below.

Furin activity and its cellular expression: Furin activity: Effects of PB on furin enzymatic activity were evaluated using a SensoLyte Rh110 Furin Activity Assay Kit (AnaSpec, Fremont, CA) in accordance with the manufacturer's protocol. Briefly, PB at 5.0 and 10 μg/ml concentrations were mixed with furin recombinant protein for 15 minutes, followed by the addition of fluorogenic Rh110 furin substrate. The samples were incubated for 1 hour at 22° C. and the fluorescence was recorded at extension/emission=490/520 nm with a fluorescence spectrometer (Perceptive Biosystems Cytofluor 4000). Data are presented as a % of control without PB addition (mean+/−SD, n=6).

Furin expression: Monolayers of A549 cells in 96-well plates were exposed to PB at 5.0 and 10 μg/ml concentrations for 48 hours. Cell layers were then washed with 1×PBS and fixed by incubation with 3% paraformaldehyde/0.5% Triton X-100 for 1 hour at 4° C. After four washing cycles with 1×PBS, cell layers were treated overnight with 1% bovine serum albumin (Rockland, CA) in 1×PBS at 4° C. Furin expression was analyzed by immunochemical ELISA assay using rabbit polyclonal anti-human furin primary antibodies (1:5000 dilution) (Invitrogen, CA) and polyclonal secondary antibodies conjugated with HRP (1:5000 dilution) (Rockland, CA). Nonspecific antibody-binding values were determined as HRP retention in samples not exposed to specific primary antibodies. Specific antibody binding was determined after subtraction of averaged nonspecific binding values from total binding value. Data are presented as a % of control without PB addition (mean+/−SD, n=6).

hACE2 activity and binding assays: Effect of PB on the activity of isolated hACE2 protein was examined using ACE2 Activity Screening Assay Kit (BPS Bioscience, San Diego, CA) according to the manufacturer's protocol. Briefly, 5.0 and 10 μg/ml of PB were added to ACE2 protein (200 mU/ml) for 15 minutes at 22° C., followed by addition of ACE2 fluorogenic substrate (10 μM) and incubation for 1 hour at 22° C. The positive control contained only ACE2 enzyme, and the negative control additionally contained 10% DMSO. The fluorescence was recorded at extension/emission=535/595 nm using a fluorescence spectrometer (Tecan Group Ltd., Switzerland). Data are presented as a % of control without PB addition (mean+/−SD, n=6).

Effect of PB on binding to the hACE2 receptor was examined using an ACE2 Inhibitor Screening Assay Kit (BPS Bioscience, San Diego, CA) according to the manufacturer's protocol. Briefly, plate with immobilized hACE2 receptors (1.0 μg/ml) were incubated with PB at 5.0 and 10 μg/ml concentrations for 1 hour at RT. The positive control contained 55% DMSO. After incubation, the plate was washed three times with washing buffer, blocked with blocking buffer for 1 hour, and incubated with antibody against hACE2 at 1:500 dilution for 1 hour, subsequently being washed four times, blocked with blocking buffer, and incubated with HRP-conjugated secondary antibody at 1:1000 dilution also for 1 hour. The chemiluminescence was assessed using ECL reagent kit and fluorescence spectrometer (Tecan Group Ltd., Switzerland). Data are presented as a % of control without PB addition (mean+/−SD, n=6).

Neuropilin-1 cellular expression assay: Monolayers of A549 cells in 96-well plates were exposed to PB at 5.0, 10, and 20 μg/ml concentrations for 48 hours. Cell layers were then washed with 1×PBS and fixed by incubation with 3% paraformaldehyde in PBS/0.5% Triton X-100 for 1 hour at 4° C. After four washing cycles with 1×PBS, cell layers were treated overnight with 1% bovine serum albumin (Rockland, CA) in 1×PBS at 4° C. NRP-1 expression was analyzed by immunochemical ELISA assay using rabbit polyclonal anti-human NPR-1 primary antibodies (1:5000 dilution) (Invitrogen, CA) and polyclonal secondary HRP-conjugated antibodies (1:5000 dilution) (Rockland, CA). Nonspecific antibody-binding values were determined as HRP retention in samples not exposed to specific primary antibodies. Specific antibody binding was determined after subtraction of averaged nonspecific binding values from total binding value. Data are expressed as a % of control without PB addition (mean+/−SD, n=6).

In vitro RdRp activity: In vitro RdRp activity was examined using a SARS-CoV-2 RNA Polymerase Assay Kit (ProFoldin, Hudson, MA) according to the manufacturer's protocol. Briefly, 0.5 μl of 50× recombinant RdRp was incubated with 2.5 μl of 50× buffer and 21 μl of PB at 5.0, 10, 25, and 100 μg/ml concentrations for 15 minutes at RT, followed by the addition of master mix containing 0.5 μl of 50×NTPs and 0.5 μl of 50× template (as a single-stranded polyribonucleotide). The reaction (25 μl) was incubated for 2 hours at 34° C. and then stopped by addition of 65 μl of 10× fluorescence dye, and the fluorescence signal was recorded within 10 minutes at extension/emission=488/535 nm using a fluorescence spectrometer (Tecan, Group Ltd., Switzerland). Positive control contained 100 μg/ml remdesivir. Results are expressed as a % of control without PB addition (mean+/−SD, n=6).

Viability: Cell viability assay was performed using MTT substrate. Briefly, 40×10³ A549 cells per well were treated with different concentrations of PB for up to 48 hours. Next, wells were washed with 1×PBS and complete growth medium supplemented with 5 mg/ml MTT was added, followed by incubation for 4 hours at 37° C. Next, the culture medium was aspirated and 100 μl of methanol was added. The absorbance was assessed at 570 nm with fluorescence spectrometer (Molecular Devices, San Jose, CA). Data are presented as a % of control without PB addition (mean+/−SD, n=6).

Western blot: A495 cells were lysed with lysis buffer [R IPA buffer plus 1× Protease Inhibitor (ThermoFisher Scientific, Waltham, MA)]. The protein estimation was performed with Dc Protein Assay (Bio-Rad, Hercules, CA). A 45 μg/well of protein was separated on 8-16% gradient SDS-PAGE gels and transferred to a PVDF membrane. Detection was performed with antibodies against cathepsin L at 1:200 dilution (Santa Cruz Biotechnology, Santa Cruz, CA) and against β-actin at 1:1000 dilution (Cell Signaling, Danvers, MA).

Statistical analysis: Data for all experiments are presented as an average value and standard deviation from at least three independent experiments. Comparison between different samples was done by a two-tailed T-test using the Microsoft Office Excel program. Differences between samples were considered significant at p values lesser than 0.05.

### Results

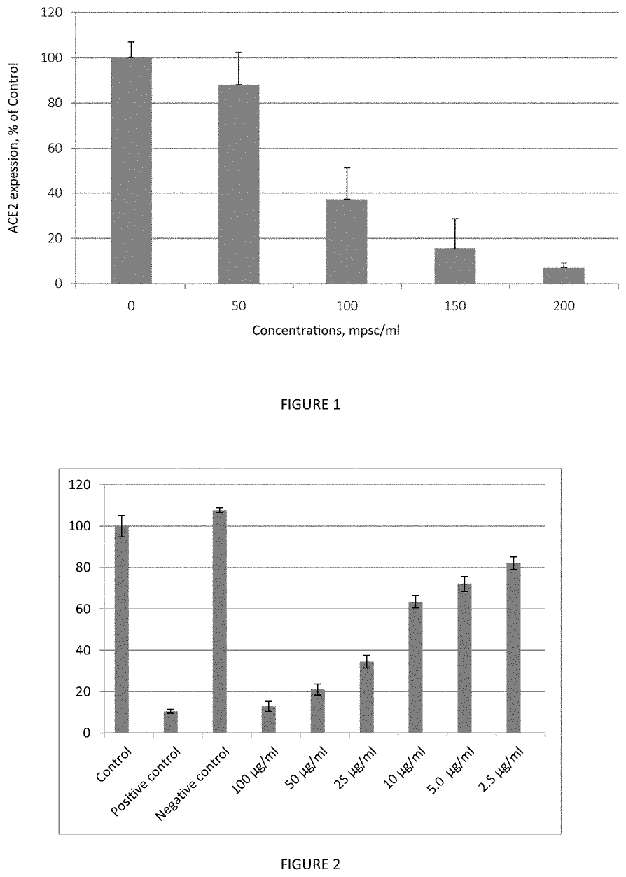

Efficacy of specific micronutrient combination on ACE2 expression in small alveolar epithelial cells. The results on FIG. 1 show that the micronutrient combination tested in this study was effective in significantly decreasing cellular expression of ACE2 receptor on human alveolar epithelial cells resulting in its 92% inhibition. Changes in ACE2 expression presented as % of control.

Effects of specific micronutrient combination (Formula 1) on RBD binding to ACE2 receptor: Binding of the RBD domain on the spike of coronavirus is the necessary step in its infectivity. FIG. 2 shows the concentration dependent effect of Formula 1 on the attachment of RBD spike domain of the SARS-CoV-2 surrogate virus to its cellular receptor ACE2. The results show that this specific micronutrient combination was effective in inhibiting viral binding by 97% compared to negative control when applied at 100 mcg/ml concentration. Its strong efficacy in preventing viral spike binding was observed already at a low concentration of 2.5 mcg/ml, causing about 20% binding inhibition. Changes in binding are expressed as % of Control. Positive control—binding inhibited, Negative control—no binding inhibition.

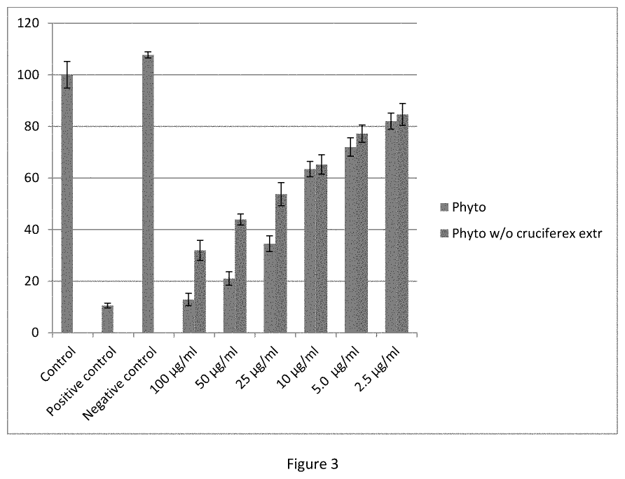

Effects of a change of one component in the Formula 1 on RBD binding to ACE2 receptor: FIG. 3 shows the results show that Formula 1 without one of its component (cruciferous plant extract) is similarly effective to the original formula. Only at concentrations of 50 and 100 mcg/ml. the blocking of RBD binding was lower than obtained with the original formulation Changes in binding are expressed as % of Control. Positive control—binding inhibited, Negative control—no binding inhibition.

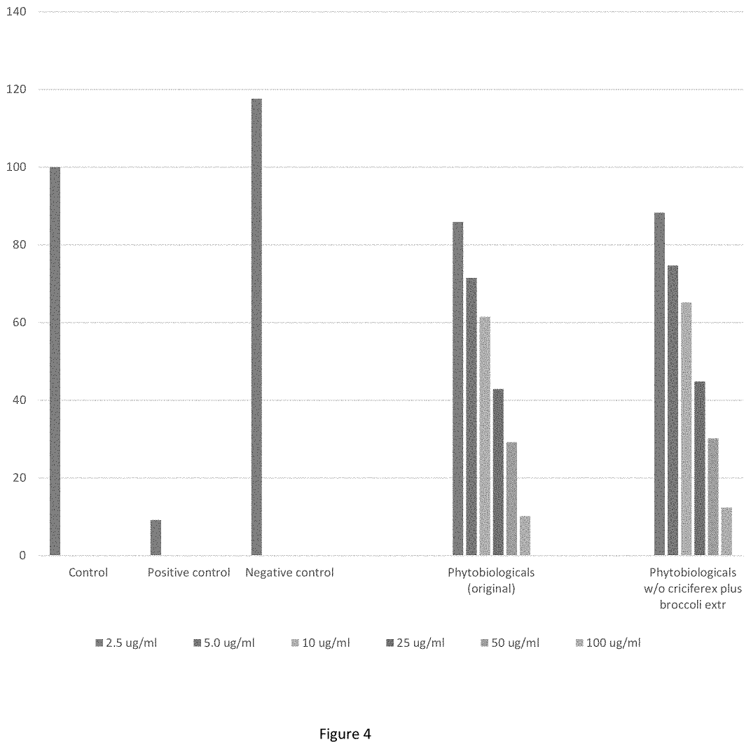

Effects of a replacing of one component in the Formula 1 with broccoli extract on RBD binding to ACE2 receptor: FIG. 4 shows that a replacement of cruciferous plant extract with broccoli extract does not affect RBD binding efficacy compared to the original Formula 1. Changes in binding are expressed as % of Control. Positive control—binding inhibited, Negative control—no binding inhibition.

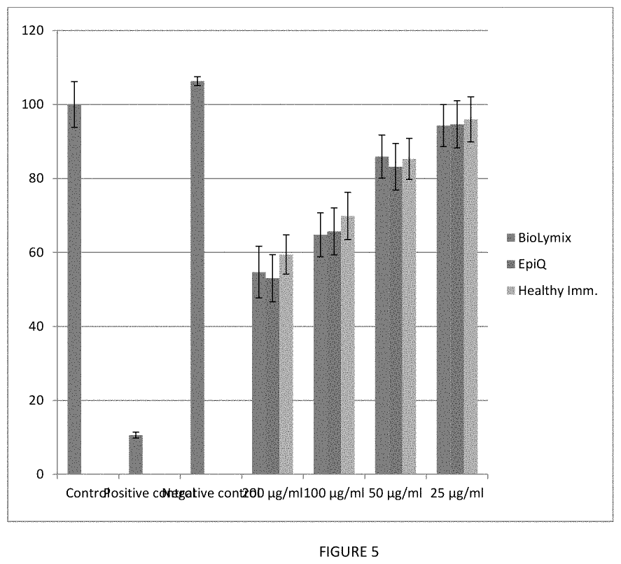

Effects of three different micronutrient combinations (Formula 2, 3 and 4) on RBD binding to ACE2 receptor: FIG. 5 shows that three other tested combinations of micronutrients designated as Formulas 2, 3 and 4 have also concentration dependent effect of RBD binding to ACE2 receptor. The RBD binding was inhibited by about 50%—at the highest tested concentration of 200 mcg/ml. Positive control indicates 100% blockage of spike RBD, Negative control—no binding inhibition. The individual compounds were also tested. The results in Table 3 show that various individual natural compounds have a profound effect on coronavirus COVID-2 spike RBD binding to ACE2 receptor with RBD inhibition between 75%-100%.

TABLE 3
The effects of individual compounds on viral RBD binding to ACE2 receptor.

| | | Binding to | |
|---|---|---|---|
| | | RBD (% of | |
| | | control) as | |
| | | a potential | |
| | | inhibitor of | |
| | | viral spike | Physiological |
| Class of compounds | Compound | attachment | dose range |
| Polyphenols (0.1 mg/ml) | Tannic acid | 79 | 1 mg-200 mg |
| Flavinoids | Curcumin | 100 | 1 mg-10,000 mg |
| | Hesperidin | 90 | 1 mg-2,000 mg |
| | (+) Epigallo cathechin gallate (EGCG) | 87 | 1 mg-5,000 mg |
| | (−)-gallocatechin gallate | 75 | 1 mg-5,000 mg |
| | Brazilin | 100 | 1 mg-1,000 mg |
| Plant extracts (0.1 mg/ml) | Tea extract (85% catechins) | 88 | 0.1 mg-10,000 mg |
| | Tea extract (85% theaflavins) | 100 | 0.1 mg-10,000 mg |
| | Theaflavin 3′3 di-gallate | 99 | 1 mg-30,000 mg |
| Volatile oils (5%) | Clove oil | 99 | 1 mg-400 mg |
| | Eugenol from clove oil | 100 | 1 mg-300 mg |
| | Oregano oil | 100 | 1 mg-1,000 mg |
| | Carvacrol (from oregano oil) | 100 | 1 mg-500 mg |
| | Cinnamon oil | 75 | 1 mg-1,000 mg |
| | Trans-trans-cinnamaldehyde | 76 | 1 mg-4,000 mg |
| | Thyme oil | 78 | 0.1 mg-100 mg |
| Fatty acids | linolenic acid, eicosapentaenoic acid, | 98-99 | 1 mg-8,000 mg |
| Polyunsaturated: | docosahexaenoic acid, linoleic acid | | |
| Fatty acids | oleic acid | 91 | 1 mg-20,000 mg |
| Monounsaturated; | Petroselinic acid | 88 | 1 mg-4,000 mg |
| Lipid soluble vitamins | Vitamin A (retinol) | 97 | 10 IU-50,000 IU |

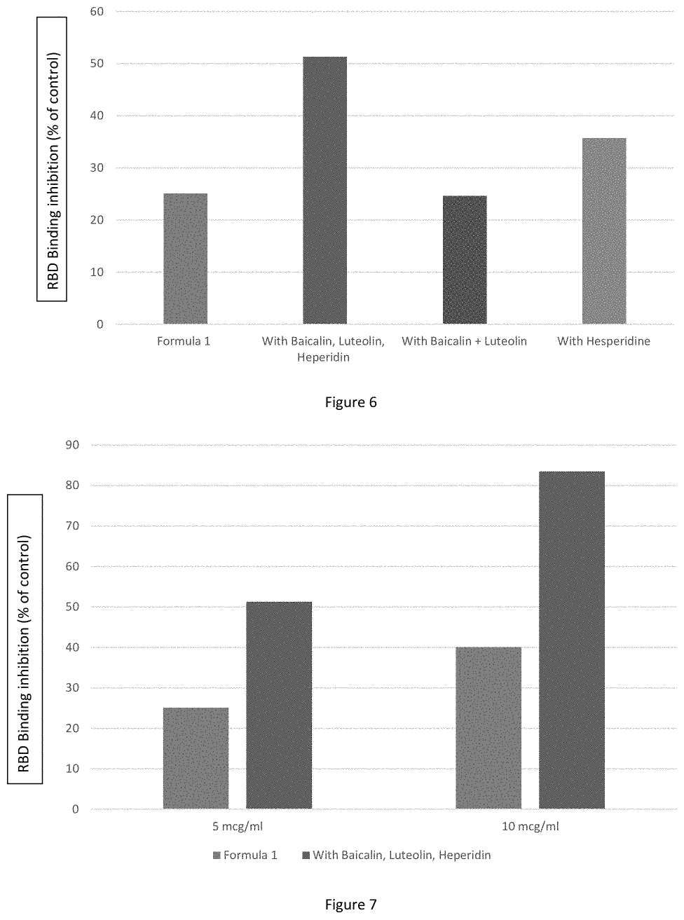

FIG. 6 shows that Formula 1 used at 100 mcg/ml concentration can inhibit RBD binding toy ACE2 receptor b5, 97%. RBD binding efficacy of lower concentrations of Formula 1 can be enhanced by combining it with specific micronutrients. These results show that the RBD binding efficacy of Formula 1 used at 5 mcg/ml can be significantly enhanced by combining it with three micronutrients (Baicalin, Luteolin and Hesperidin) at 50 meg/nil each. As such, the efficacy of RBD binding inhibition to ACE2 by Formula 1 applied at 5 mcg/ml can be increased by adding these three micronutrients from 25.1% to 51.3%. These results suggest synergistic effects of these micronutrients. When these three micronutrients were tested individually, they showed only minimal enhancement in blocking the RBD binding to ACE2 receptor.

FIG. 7 shows enhanced inhibition of RBD binding to ACE2 receptor by Formula 1 in combination with specific micronutrients. The results of Baicalein+Luteolin+Hesperidin were applied together with: 5 mcg/ml concentration of Formula 1. This increased binding inhibitory effect from 25.1% to 51.3%. 10 mcg/ml concentration of Formula 1. This increased binding inhibitory effect from 40.1% to 83.5%.

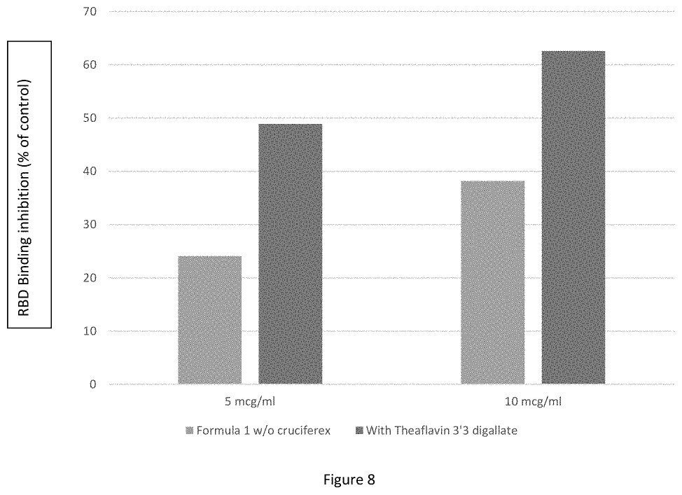

FIG. 8 shows the test of the efficacy of Formula 1 w/o cruciferex can be enhanced by its combination with theaflavin for its inhibitory effect on RBD binding to ACE2 receptor. The results further show Theaflavin digallate combined together with Formula 1 w/o cruciferex enhances the efficacy of RBD binding inhibition. At 5 mcg/ml concentration of Formula 1 w/o cruciferex the inhibitory effect increased from 24.1% to 48.9%. At 10 mcg/ml concentration of Formula 1 w/o cruciferex the inhibitory effect increased from 38.2% to 62.6%.

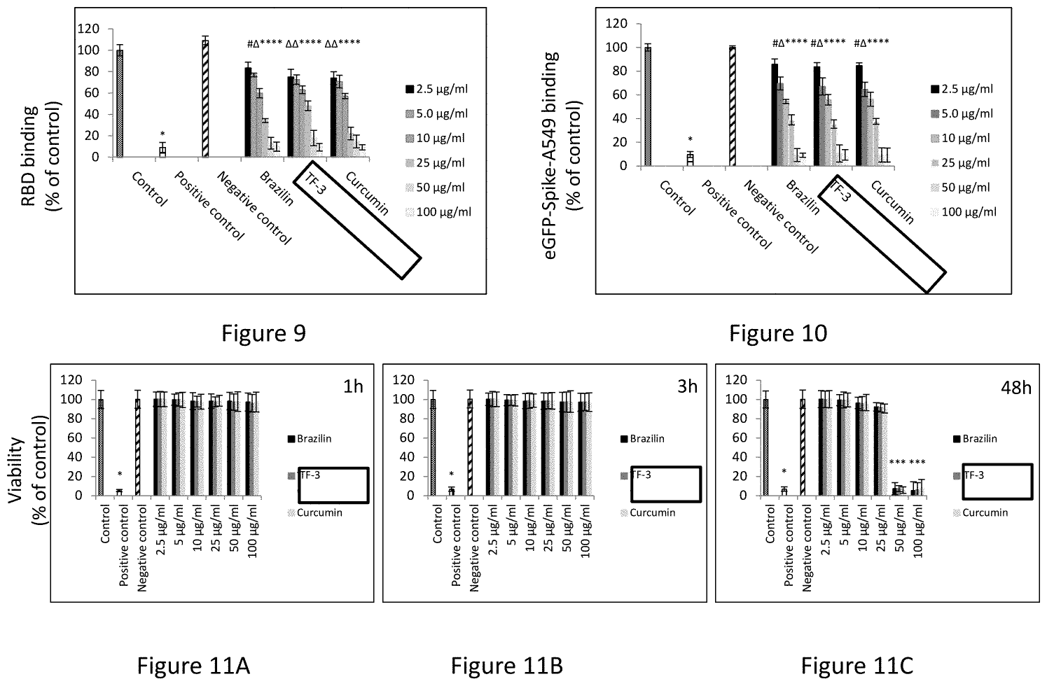

Efficacy of phenolic compounds and plant extracts in preventing binding of RBD sequence of SARS-CoV-2 with hACE2 receptor: We investigated the ability of several classes of polyphenols to inhibit the binding of the RBD sequence of the SARS-CoV-2 spike protein to the hACE2 receptor, taking a two-stage approach. In the first approach we screened the capacity of 56 polyphenols and plant extracts, to inhibit binding of HRP-conjugated RBD-SARS-CoV-2 spike protein to the immobilized hACE2 receptor. As presented in Tables 4 and 5, three polyphenols: brazilin, TF-3, and curcumin showed the highest inhibitory effect at 100 μg/ml concentration. Moreover, the inhibitory effect of these most effective polyphenols, i.e., brazilin, TF-3, and curcumin, was dose dependent, ranging from 20% to 100% at 2.5-100 μg/ml, respectively (FIG. 9).

In the second approach, we incubated A549 cells expressing SARS-CoV-2 spike protein with these three selected polyphenols for 1 h and then exposed them to the soluble hACE2 receptor. In this experiment, we also observed dose-dependent interference ranging from 15% to 100% at 2.5-100 μg/ml, respectively, which corresponded to previously obtained results (FIG. 10). A cell viability test revealed that short-term incubation (i.e., up to 3 h) with these polyphenols at concentrations up to 100 μg/ml showed no cytotoxicity. However, with incubation time extended to 48 hours at doses of 50 μg/ml and above, decreased cell viability was noticed (FIG. 11A, FIG. 11B, FIG. 11C).

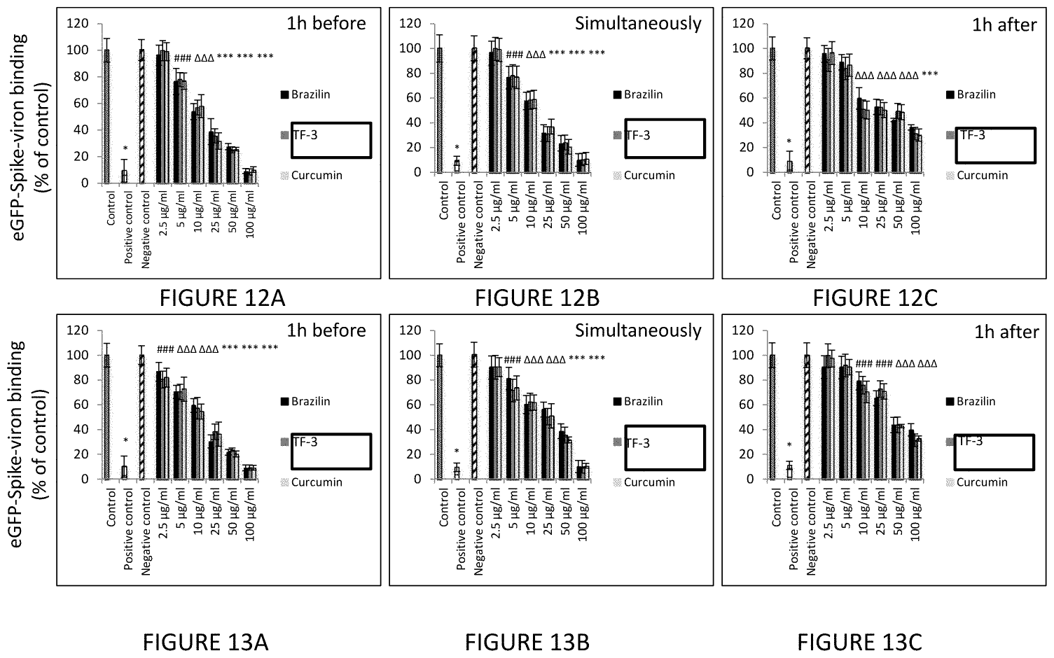

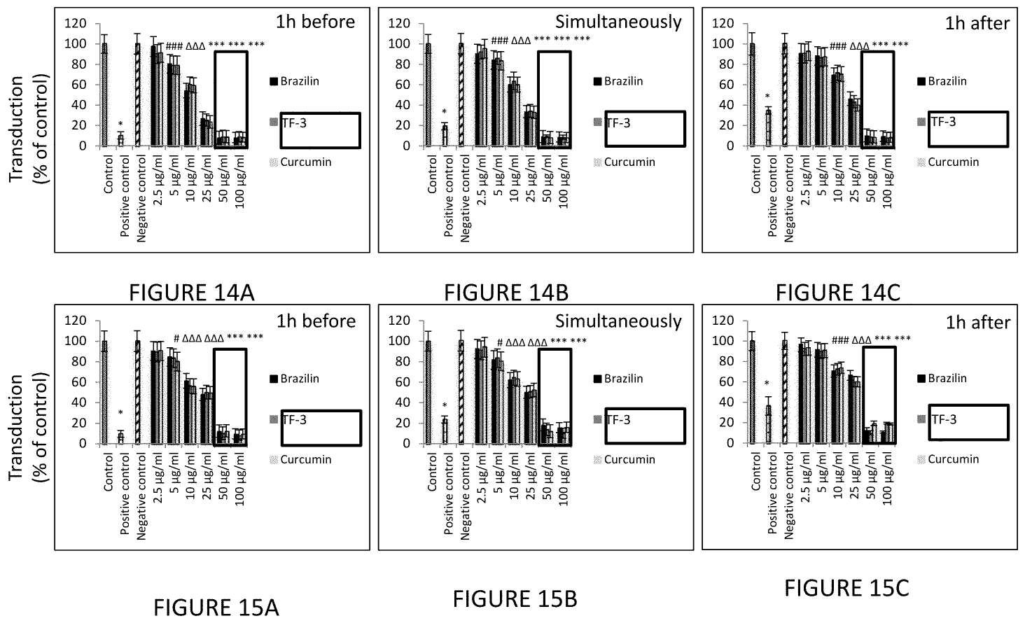

Effects of brazilin, theaflavin-3,3′-digallate and curcumin on binding and cellular entry of SARS-CoV-2 pseudo-virions: In subsequent experiments, we tested whether observed inhibitory effects of brazilin, TF-3, and curcumin, on RBD binding to hACE2, will persist when using SARS-CoV-2 viral particles. In these tests we used pseudo-virions enveloped with SARS-CoV-2 spike protein, and applied three different patterns, as follows: 1) SARS-CoV-2 virions carrying the genes for GFP-luciferase and pseudo-typed with the spike protein were incubated with selected polyphenols for 1 h before being added to hACE2/A549 cells, 2) SARS-CoV-2 virions carrying the genes for GFP-luciferase and pseudo-typed with the spike protein were added simultaneously to hACE2/A549 cells, and 3) SARS-CoV-2 virions carrying the genes for GFP-luciferase and pseudotyped with the spike protein were added to hACE2/A549 cells, and 1 h after polyphenols were applied to the hACE2/A549 cells. Binding efficacy for each application pattern was evaluated after either 1 h or 3 h of incubation with the hACE2/A549 cells. Also, we evaluated the efficacy of these polyphenols after 48 h post-infection, with or without spin-inoculation. Binding efficacy experiment revealed that brazilin, TF-3, and curcumin inhibit, in dose-dependent fashion, binding of SARS-CoV-2 spike protein pseudo-typed virions to hACE2/A549, regardless of exposure time and application pattern. This experiment also showed significant inhibition by these polyphenols, starting from 5.0 μg/ml, when 1 h incubation was allowed (FIG. 12A, FIG. 12B, FIG. 12C). With incubation extended to 3 h, significant inhibition was observed from 2.5 μg/ml when SARS-CoV-2 virions were incubated with selected polyphenols for 1 h before being added to hACE2/A549 cells. When SARS-CoV-2 virions were added simultaneously with selected polyphenols to hACE2/A549 cells, significant inhibition was noticed from 5.0 μg/ml, and from 10 μg/ml when selected polyphenols were applied in hACE2/A549 cells 1 h after SARS-CoV-2 virions were applied (FIG. 13A, FIG. 13B, FIG. 13C). The experiments, in which incubation was extended to 48 h and whether or not spin-inoculation was applied, also revealed that brazilin, TF-3, and curcumin inhibit, in dose-dependent fashion, binding of SARS-CoV-2 spike protein pseudo-typed virions A549 to hACE2/A549 at non-toxic concentrations (i.e., 5.0-25 μg/ml). Inhibition ranged from 20% to 80% when spin-inoculation was not introduced, and from 20% to 40% when spin-inoculation was introduced (FIG. 14A). When spin-inoculation was not applied, significant inhibition was observed from 5.0 μg/ml concentration when SARS-CoV-2 spike pseudo-virions were either incubated with selected polyphenols 1 h before hACE2/A549 cell exposure, or when SARS-CoV-2 spike pseudo-virions were added simultaneously with the tested polyphenols (FIG. 14b and FIG. 14C). When the tested polyphenols were added 1 h after SARS-CoV-2 pseudo-virions were applied, significant inhibition was noticed from 10 μg/ml concentration. When the viral binding to the hACE2/A549 cells was forced by application of spin-inoculation, significant inhibition was observed from 5.0 μg/ml when SARS-CoV-2 virions incubated with curcumin for 1 h before being added to hACE2/A549 cells or when SARS-CoV-2 spike pseudo-virions were added simultaneously with curcumin. When SARS-CoV-2 virions were incubated with brazilin or TF-3, the inhibitory effect was observed from 10 μg/ml concentration. When test polyphenols were added 1 h after SARS-CoV-2 virions were applied, significant inhibition was noticed from 10 μg/ml concentration (FIG. 15A, FIG. 15B and FIG. 15C).

Also, our further experiment, where A549 cells expressing SARS-CoV-2 spike protein pseudo-typed virions were pre-incubated with the tested polyphenols, and then layered for 4 h on hCE2/A549, the cells showed a significantly decreased attachment. Incubation with brazilin at 25 μg/ml decreased the fusion by 40%, with TF-3 at 10-25 μg/ml by 40% to 70%, and with curcumin at the same concentrations, i.e., 10-25 μg/ml, by 70% to 95%. The results were consistent with previously obtained sets of data.

TABLE 4
Binding of various classes of phenolic
compounds with RBD of SARS-CoV-2.

| | | Physiological levels |
|---|---|---|
| Tested polyphenols and | Binding with RBD | for Micronutrient |
| alkaloids (0.1 mg/ml) | (% of control ± SD) | composition |
| Phenolic acids | | |
| Gallic acid | 18.3 ± 4.5 | 1 mg-1500 mg |
| Tannic acid | 79.4 ± 2.3 | 1 mg-200 mg |
| Curcumin | 100 ± 0.2 | 1 mg-10,000 mg |
| Chlorogenic acid | 25.5 ± 2.5 | 1 mg-4,000 mg |
| Rosmarinic acid | 22.5 ± 3.8 | 1 mg-2,000 mg |
| Flavonoids | | |
| Fisetin | 22.4 ± 1.9 | 1 mg-2,000 mg |
| Quercetin | 22.4 ± 6.5 | 1 mg-5,000 mg |
| Morin | 30.5 ± 5.8 | 1 mg-1,000 mg |
| Myricetin | 45.5 ± 5.4 | 1 mg-1,000 mg |
| Kaemferol | 15.6 ± 2.9 | 1 mg-2,000 mg |
| Rutin | 20.6 ± 6.3 | 1 mg-4,000 mg |
| Luteolin | 10.4 ± 4.7 | 1 mg-100 mg |
| Baicalein | 22.5 ± 5.1 | 1 mg-4,000 mg |
| Baicalin | 10.3 ± 2.9 | 1 mg-4,000 mg |
| Scutellarin | 8.1 ± 3.7 | 1 mg-2,000 mg |
| Naringin | 23.6 ± 6.4 | 1 mg-3,000 mg |
| Naringenin | 20 ± 5.1 | 1 mg-3,000 mg |
| Hesperidin | 90.3 ± 3.8 | 1 mg-2,0000 mg |
| Hesperetin | 42.5 ± 4.6 | 1 mg-2,000 mg |
| Apigenin | 17.1 ± 4.1 | 1 mg-1,000 mg |
| Genistein | 22.1 ± 2.8 | 1 mg-1,000 mg |
| Phloroglucinol | 69.5 ± 3.6 | 1 mg-100 mg |
| Schizandrin | 22.4 ± .3.3 | 1 mg-5,000 mg |
| Urolithin A | 31.1 ± 4.6 | 1 mg-1,000 mg |
| Punicalagin | 32.3 ± 5.9 | 1 mg-1,000 mg |
| Brazilin | 100 ± 0.1 | 1 mg-1,000 mg |
| Hispidulin | 20.1 ± 6.0 | 1 mg-1,000 mg |
| Papaverine | 1.6 ± 0.2 | 1 mg-300 mg |
| Silymarin | 30.0 ± 2.6 | 1 mg-2,000 mg |
| Procyanidin B2 | 31.1 ± 3.6 | 1 mg-1,000 mg |
| Procyanidin B3 | 32.3 ± 3.7 | 1 mg-1,000 mg |
| Stilbenes | | |
| Trans-resveratrol | 22.3 ± 2.9 | 1 mg-3,000 mg |
| Pterostillbene | 23.1 ± 2.8 | 1 mg-800 mg |
| Alkaloids | | |
| Palmatine | 40.4 ± 6.1 | 1 mg-2,000 mg |
| Berberine | 17.3 ± 2.7 | 1 mg-2,000 mg |
| Cannabidiol | 1.4 ± 0.3 | 1 mg-2,000 mg |
| Castanospermine | 8.2 ± 2.3 | 1 mg-1,000 mg |
| Usnic acid | 22.0 ± 3.4 | 1 mg-800 mg |
| Malic acid | 1.2 ± 3.7 | 1 mg-4,000 mg |
| Terpenes | | |
| D-limonene | 27.2 ± 6.4 | 1 mg-1500 mg |
| Carnosic acid | 27.1 ± 5.1 | 1 mg-700 mg |

TABLE 5
Binding of selected plant extracts and their major components with RBD of SARS-COV-2

| Tested plant extracts and their main active | Binding with RBD (% of | |
|---|---|---|
| compounds (0.1 mg/ml) Tea extract (85% catechin | control ± SD) | |
| standardized) | 88.3 ± 3.7 | Physiological dose range |
| (+)-gallocatechin | 69.5 ± 2.8 | 1 mg-5,000 mg |
| (−)-catechin gallate | 37.4 ± 4.7 | 1 mg-5,000 mg |
| (−)-gallocatechin gallate | 75.4 ± 5.6 | 1 mg-5,000 mg |
| (−)-gallocatechin | 73.5 ± 6.7 | 1 mg-5,000 mg |
| (+)-epigallocatechin gallate | 87.5 ± 6.8 | 1 mg-5,000 mg |
| Tea extract (85% theaflavins standardized) | 100 ± 0.3 | 0.1 mg-10,000 mg |
| Theaflavin | 27.3 ± 1.4 | 1 mg-30,000 mg |
| Theaflavin-3,3′-digallate | 100 ± 0.1 | 1 mg-30,000 mg |
| Broccoli extract | 28.6 ± 2.6 | 1 mg-5,000 mg |
| L-sulforaphane | 30.2 ± 3.6 | 1 mg-5,000 mg |
| Andrographis paniculata extract | 18.4 ± 1.8 | 1 mg-20,000 mg |
| Andrographolide | 22.1 ± 2.5 | 1 mg-10,000 mg |
| Licorice extract | 18.3 ± 3.6 | 1 mg-20,000 mg |
| Glycyrrhizic acid | 22.2 ± 2.3 | 1 mg-5,000 mg |

FIGS. 9, 10 and 11a and 11b. Binding of RBD-spike protein of SARS-CoV-2 to human ACE2 receptor. FIG. 9 shows dose-dependent binding of RBD-SARS-CoV-2 to immobilized hACE2 receptor. Control—0.025% DMSO, positive and negative controls were provided by the manufacturer; data are presented as % of control±SD. FIG. 10 shows dose-dependent binding of A546 cells expressing SARS-CoV-2 eGFP-spike protein, in the presence of indicated polyphenols at different concentrations, to soluble hACE2 receptor. Control—0.25% DMSO; positive and negative controls were provided by the manufacturer; data are presented as % of control±SD. FIG. 11A, FIG. 11B and FIG. 11C shows viability of A549 cells, positive control-100% dead cells, negative control—addition-free sample; TF-3—theaflavin-3,3′-digallate; #p≤0.05, ×p≤0.01, p≤0.001.

FIG. 12A, FIG. 12B and FIG. 12C shows binding of SARS-CoV-2 pseudo-virion to human ACE2 receptor. Dose-dependent binding of SARS-CoV-2 spike protein-encapsulated pseudo-virions to A549 cells stably overexpressing human ACE2 receptor evaluated after 1 h incubation. FIG. 13A, FIG. 13B and FIG. 13C shows dose-dependent binding of SARS-CoV-2 spike protein-encapsulated pseudo-virions to A549 cells stably overexpressing hACE2 receptor evaluated after 3 h incubation. Data are presented as % of control±SD; control—0.025% DMSO, positive and negative controls were provided by the manufacturer; TF-3—theaflavin-3,3′-digallate #p O 0.05, Δp O 0.01, p O 0.001.

FIG. 14A, FIG. 14B and FIG. 14C show SARS-CoV-2 eGFP-luciferase-pseudo-virion cellular entry. Attachment and entry of SARS-CoV-2 pseudo-virions with encapsulated eGFP-luciferase spike protein was evaluated without spinfection after 48 h incubation. FIG. 15A, FIG. 15B and FIG. 15C show attachment and entry of SARS-CoV-2 pseudo-virions with encapsulated eGFP-luciferase spike protein was evaluated with spinfection after 48 h incubation. Data are presented as % of control±SD; TF-3—theaflavin-3,3′-digallate #p≤0.05, Δp≤0.01, p O 0.001. Control—0.025% DMSO, positive control—bald SARS-CoV-2 eGFP-luciferase-pseudo-virions, negative control—ΔG-luciferase rVSV pseudo-typed particles; red fame—concentrations that showed 85-100% cytotoxicity.

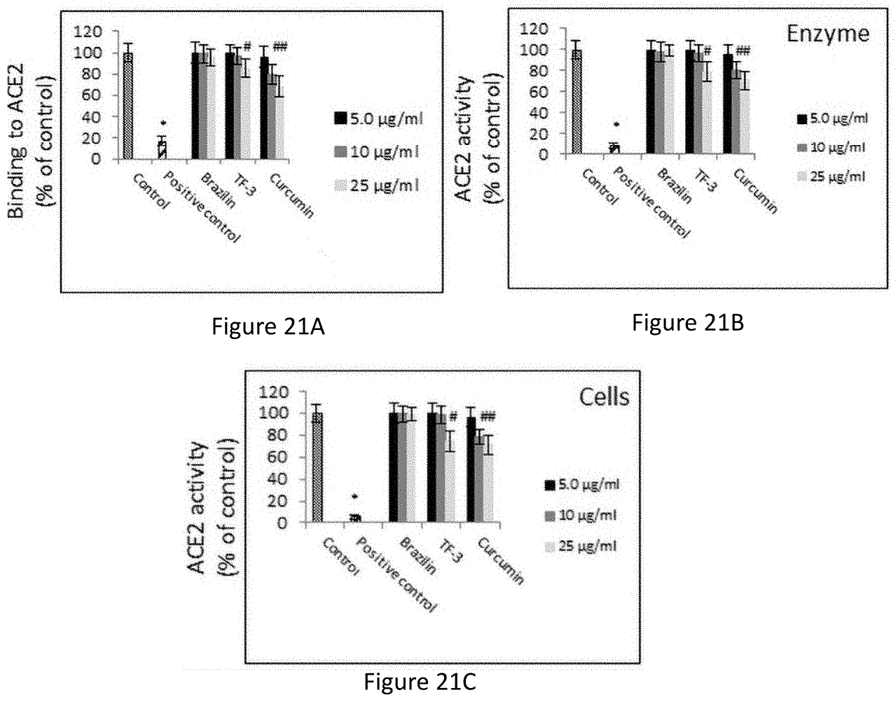

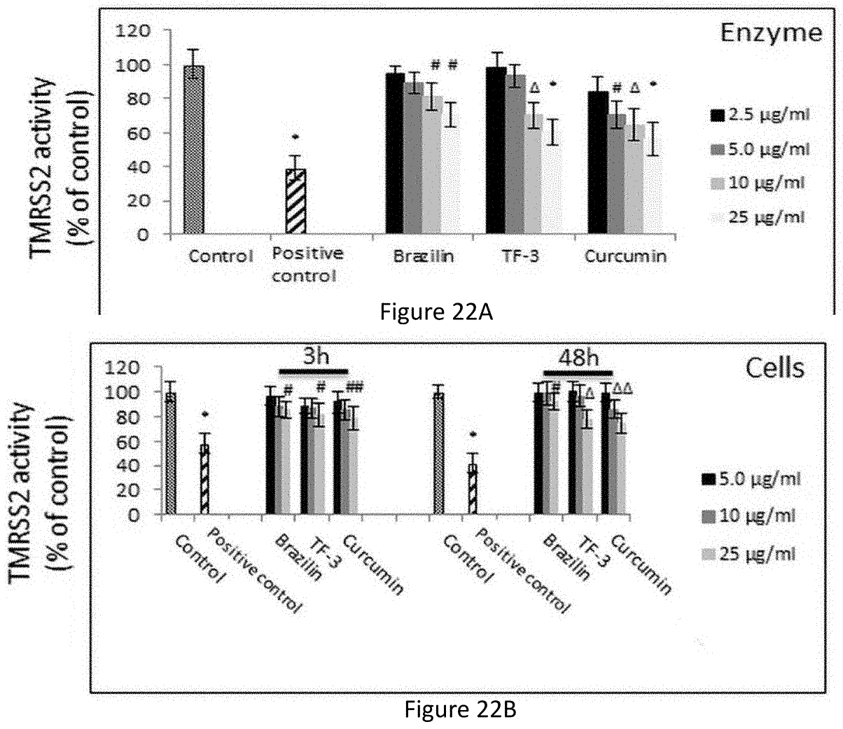

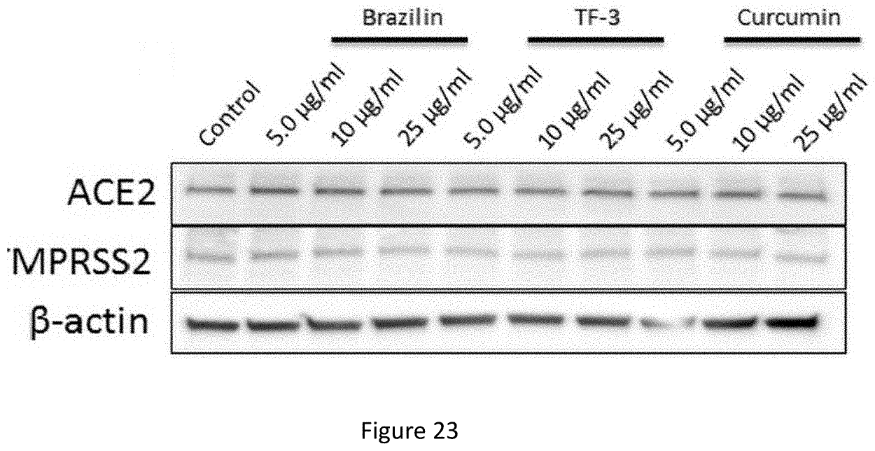

Effect of brazilin, theaflavin-3,3′-digallate and curcumin on cellular proteases involved in entry and endosomal egress of SARS-CoV-2 pseudo-virions: The crucial step in the SARS-CoV-2 virions internalization involves the cognate ACE2 receptor. Therefore, we checked whether or not brazilin, TF-3, and curcumin affect binding to and activity of the ACE2 molecule itself. Our results showed (FIG. 21A, FIG. 21B and FIG. 21C) that brazilin does not bind to ACE2 directly, in contrast to TF-3 and curcumin, which showed binding efficacy at 25 μg/ml and at 10-25 μg/ml, respectively. In addition, we observed minor 20%-30% inhibition of ACE2 activity in both cell-free and cell-based assays with TF-3 at 25 μg/ml and curcumin at 10-25 μg/ml, respectively, and no effects with brazilin. Binding of indicated polyphenols at different concentrations to hACE2 receptor. Data are presented as % of control±SD; control—0.025% DMSO, positive control—50% DMSO. Activity of recombinant hACE2 upon treatment with indicated polyphenols at different concentrations. Activity of cellular hACE2 upon treatment with indicated polyphenols at different concentrations. Data are presented as % of control±SD; p 0.001. Control—0.025% DMSO, positive control—10% DMSO. FIG. 22A and FIG. 22B shows activity of recombinant TMPTSS2 upon treatment with indicated polyphenols at different concentrations. Data are presented as % of control±SD; #p 0.05, A p 0.01, p 0.001. Control-0.025% DMSO, positive control—50-100 μM camostat mesylate. FIG. 23 shows western blot analysis of hACE2 and TMPRSS2 expression in A549 cells upon treatment with indicated polyphenols with different concentration for 48 h period. Data are presented as % of control±SD; control—0.025% DMSO, TF-3—theaflavin-3,3′-digallate.

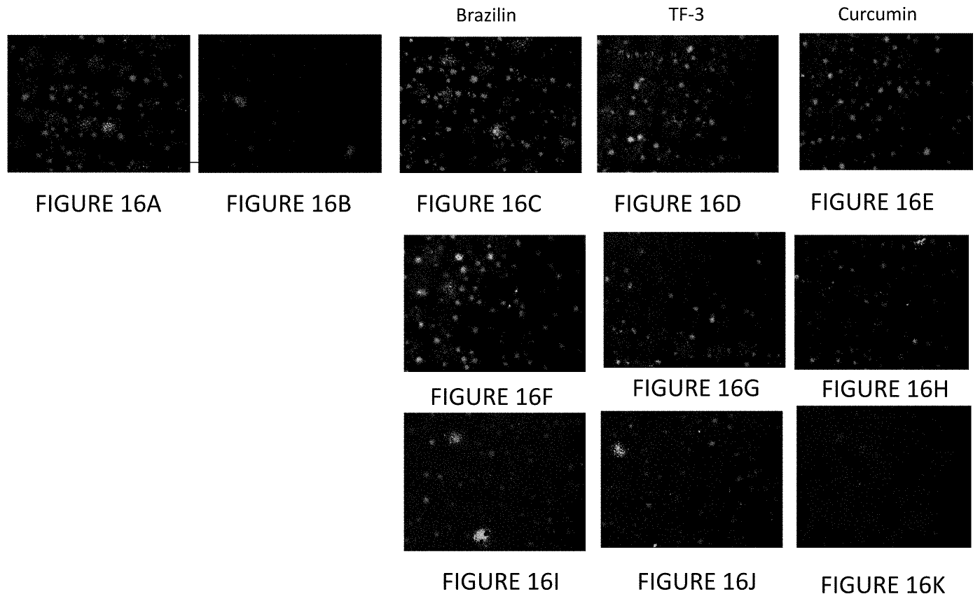

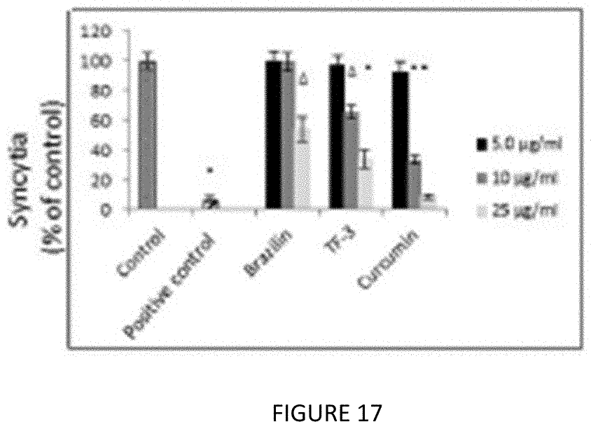

FIGS. 16A, 16B, 16C, 16D, 16E, 16F, 16H, 16I, 16J and 16K shows effect of selected polyphenols on fusion to human ACE2 receptor overexpressing A549 cells. Cell-cell fusion of A549 cells expressing eGFP spike protein with A549 cells stably expressing human ACE2 receptor. The scale bar indicates 250 μm. FIG. 17 shows quantitative analysis of formed syncytia. Experiments were done in triplicate and repeated three times. Data are presented as % of control±SD; TF-3—theaflavin-3,3′-digallate Δp O 0.01, p O 0.001. Control—0.025% DMSO, positive control—20 μg/ml anti-ACE2 antibody.

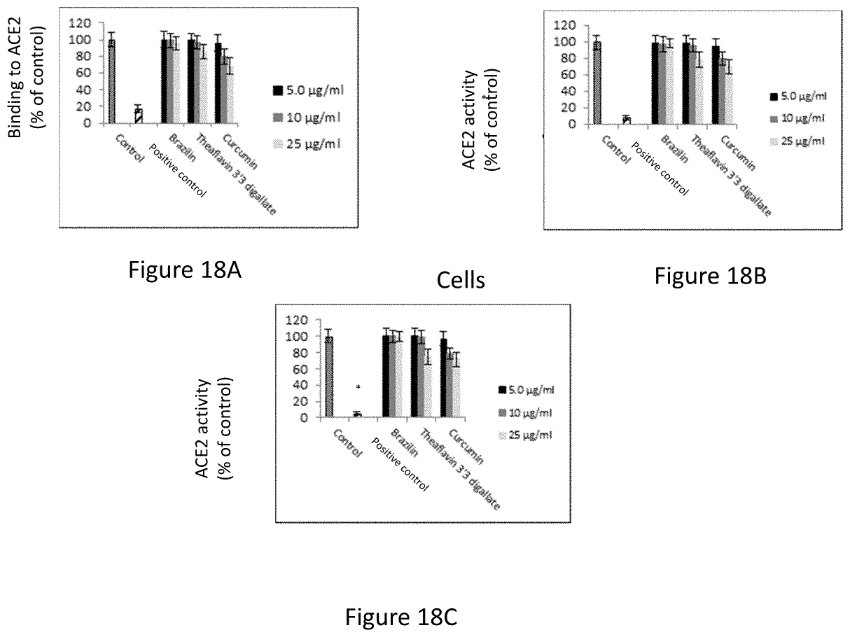

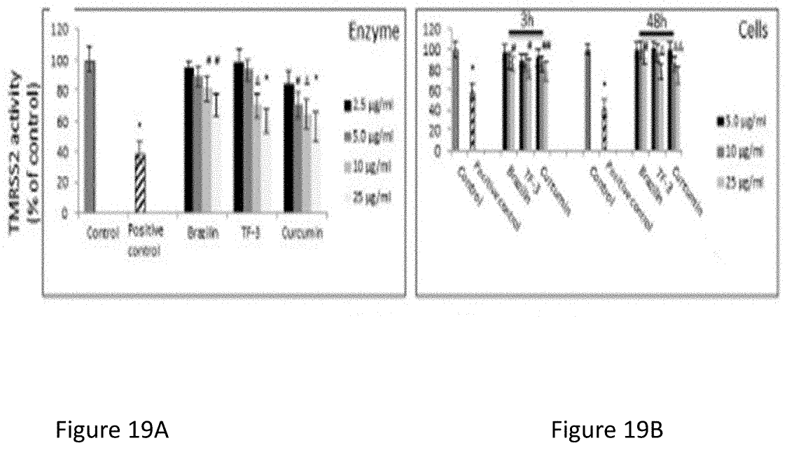

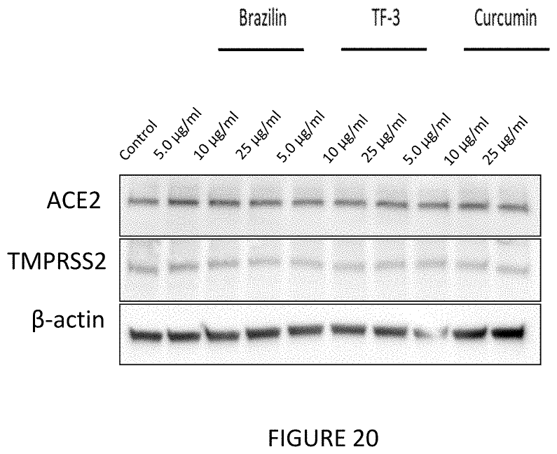

Effects of selected polyphenols on cellular membrane associated proteases. FIG. 18A, FIGS. 18B and 18C shows binding of indicated polyphenols at different concentrations to hACE2 receptor. Data are presented as % of control±SD; control—0.025% DMSO, positive control—50% DMSO. FIG. 19A shows activity of recombinant hACE2 upon treatment with indicated polyphenols at different concentrations. Activity of cellular hACE2 upon treatment with indicated polyphenols at different concentrations. Data are presented as % of control±SD; p O 0.001. Control—0.025% DMSO, positive control—10% DMSO. Activity of recombinant TMPTSS2 upon treatment with indicated polyphenols at different (FIG. 19B). Activity of cellular TMPTSS2 upon treatment with indicated polyphenols at different concentrations (right panel). Data are presented as % of control±SD; #p O 0.05, Δp O 0.01, p O 0.001. Control-0.025% DMSO, positive control—50-100 μM camostat mesylate. FIG. 20 shows western blot analysis of hACE2 and TMPRSS2 expression in A549 cells upon treatment with indicated polyphenols with different concentration for 48 h period. Data are presented as % of control±SD; control—0.025% DMSO, TF-3—theaflavin-3,3′-digallate.

In order to gain deeper insight into the mechanism by which these three polyphenols sup-press the SARS-CoV-2 virions cellular penetration, and knowing that the SARS-CoV-2 virions internalize via an endocytic pathway, but that, at the same time, host cellular proteases are involved, we checked the activity and cellular expression of TMPRSS2. As shown in FIG. 19A, significant inhibition of recombinant hTMPRSS2 activity was observed, upon 3 h treatment with brazilin and TF-3 at 10-25 μg/ml, ranging from 20-30% for brazilin, and from 30% to 40% for TF-3, whereas curcumin treatment decreased TMPRSS2 activity by about 40% to 50%. Activity of hTMPRSS2 overexpressed on A549 cells was also affected by these compounds upon 48 h treatment that followed the pattern observed in short-term experiment (i.e., 3 h treatment). Our results also showed that expression of ACE2 and TMPRSS2 at protein level was not affected (FIG. 20).

To further clarify if other components known to be involved in the SARS-CoV-2 virions' cellular penetration, we checked activity and cellular expression of cathepsin L, utilizing human recombinant enzyme and enzyme derived from lysates of A549 cells treated with the tested polyphenols. In experiment with recombinant enzymes, curcumin proved to have the most profound inhibitory effect, ranging from 40% to 50% at 1.0-2.5 μg/ml. TF-3 followed, and showed 20% to 30% inhibition at 1.0-2.5 μg/ml, but brazilin had a minor, not significant effect. In cell lysates, we observed a similar trend, although inhibition of cathepsin L required 10 times higher concentrations of curcumin, which showed 20%-45% inhibition at 5.0-25 μg/ml, and TF-3, which revealed 20-25% inhibition at 10-25 μg/ml. Brazilin caused not significant 15% decrease at 25 μg/ml. Interestingly, neither brazilin nor curcumin downregulated cathepsin L expression at protein level, in contrast to TF-3, which modestly decreased its expression by about 20% starting from 10 μg/ml concentration.

Knowing that cathepsin L is a pH-sensitive protease, we employed 20 mM ammonia chloride as a positive control to check lysosomal/endosomal pH. Our results revealed that brazilin and curcumin can increase pH to about 6.0-6.5 at 5.0-25 μg/ml, whereas TF-3 elevates pH to about 5.5-6.0 at 5.0-25 μg/ml, compared with a control that, when measured, showed approximately pH=5.0. This pattern was corroborated in the further experiment, where decreased fluorescence was observed upon treatment with these polyphenols at 5.0-25 μg/ml and acridine orange utilized as a pH sensor.

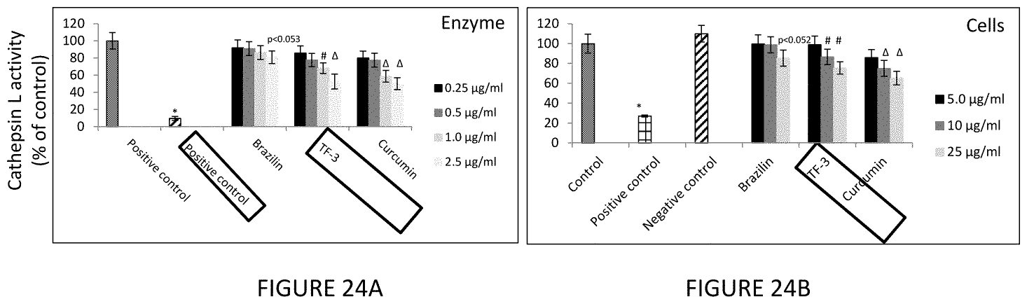

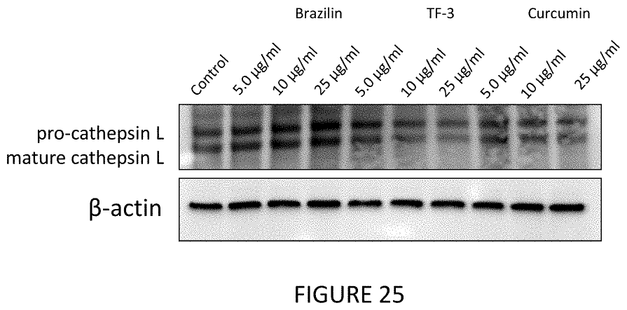

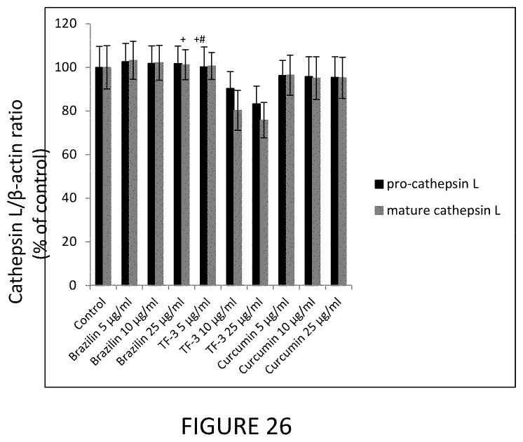

Effect of selected polyphenols on cathepsin L. FIG. 24A and FIG. 24B shows activity of purified cathepsin L enzyme upon treatment with indicated polyphenols at different concentrations. Activity of cellular cathepsin L upon treatment with indicated polyphenols at different concentrations. Data are presented as % of control±SD; Δp O 0.01, p O 0.001, +p>0.054. Control—0.025% DMSO, positive control—0.1 μM E-64. FIG. 25 shows western blot analysis of cathepsin L expression in A549 cells treated with indicated polyphenols with different concentration for 24 h. and quantified as band densitometry analysis indicating changes in protein expression (FIG. 26). Data are presented as % of control±SD; control—0.025% DMSO, TF-3—theaflavin-3,3′-digallate.

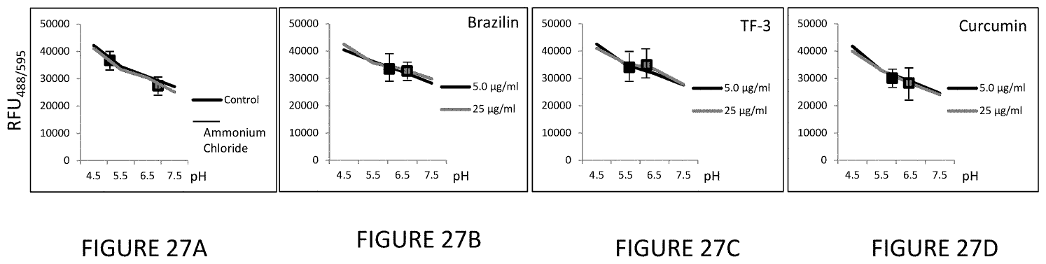

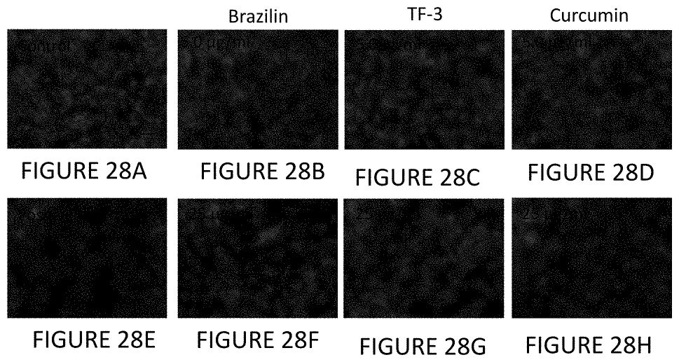

Effect of selected polyphenols on internal pH and endosome acidification. FIG. 27A, FIG. 27B, FIG. 27C and FIG. 27D show intracellular/lysosomal pH measurement. pHrodo™ Green AM dye and additional incubation for 30 min. at 37° C. Cells were then washed and fluorescence was measured at Ex/Em=535/595 nm. Intracellular pH identification was done using standard curve prepared by measuring fluorescence in the presence of standard buffers with indicated pH as described in Material and Methods section. FIG. 28A, FIG. 28B, FIG. 28C, FIG. 28D, FIG. 28E, FIG. 28F, FIG. 28G and FIG. 28H show Endosomal pH measurement in A549 cells treated with indicated polyphenols at different concentrations for 3 h at 37° C. Scale bar indicates 50 μm. Images are representative of all observed fields. Experiments were done in triplicates and repeated three times. Data are presented as % of control±SD. TF-3—theaflavin-3,3′-digallate; control—0.025% DMSO, positive control—20 mM ammonia chloride.

Previous studies based on computational modeling and virtual screenings suggest that polyphenols mediate their anti-SARS-CoV-2 activity through diverse mechanisms. For example, Wu et al. showed that theaflavin 3,3′-di-O-gallate, 14-deoxy-11,12-didehydroandrographolide, betulonal, and gnidicin exhibit high binding affinity to viral RdRp polymerase, whereas licoflavonol, cosmosiin, neohesperidin, and piceatannol target the binding between RBD of spike protein and hACE2, although it was predicted that only hesperidin would directly bind to the RBD of SARS-CoV-2 spike protein.

A study by Rehman et al. revealed that kaempferol, quercetin, and rutin were able to bind M⁻¹ at the SBP (Substrate Binding Pocket) of 3CLpro with high affinity (i.e., 10⁵-10⁶), interacting with active site residues of 3CLpro such as His⁴¹ and Cys¹⁴⁵. They also stated that the binding affinity of rutin was 1,000 times higher than that of chloroquine and 100 times higher than hydroxychloroquine. Based on the molecular docking study by Chen and Du, baicalin, scutellarin, hesperetin, nicotianamine, and glycyrrhizin have been identified as potential ACE2 inhibitors that could be used as possible anti-SARS-CoV-2 agents preventing its entry. Compounds such as baicalin, (−)-epigallocatechin gallate, sugetriol-3,9-diacetate, and platycodin D revealed high binding affinity to the PLpro (papain-like protease) molecule that generates Nsp1, Nsp2 and Nsp3 proteins involved in the viral replication process.

According to Patel et al., curcumin and its derivatives showed high binding affinity to the RBD of SARS-CoV-2, with AG (i.e., binding energy) between −10.01 to −5.33 kcal/mol. Based on a binding energy that resembles that of synthetic drugs, and also pharmacokinetic parameters, these researchers identified curcumin as a candidate for SARS-CoV-2 spike protein inhibition. Moreover, Jena et al. reported on catechin and curcumin, which have dual binding affinity, i.e., they bind to viral spike protein as well as to hACE2, although catechin's binding affinity is greater (i.e., catechin: −7.9 kcal/mol and −7.8 kcal/mol; curcumin: −10.5 kcal/mol and −8.9 kcal/mol, respectively). While these theoretical and molecular modelling approaches could identify potential applications of various molecules, the experimental proofs of their efficacy remain sparse.

Here, we provide in vitro evidence that among 56 tested phenolic compounds and plant extracts, brazilin, TF-3, and curcumin exhibited the highest binding to RBD-spike protein of SARS-CoV-2. Utilizing spike protein expressing hA549 cells we corroborated this result. By employing spike protein-enveloped pseudo-virions and different pattern of exposure, we observed in our short-term (i.e., exposure time 1 hour or 3 hours) and long-term studies (i.e., exposure time 48 hours), that all three compounds can inhibit viral attachment to the cell surface regardless of the time of exposure or incubation pattern. When the enveloped SARS-CoV-2 virions were pre-incubated with these compounds for 1 h, added simultaneously, or when the compounds were added 1 h post-infection to the cellular monolayer, their ability to bind to the ACE2 receptor and transduce was dose-dependently decreasing.

Interestingly, the same effect, although at higher but still non-toxic concentrations, was seen when SARS-CoV-2 pseudo-virions where forcibly attached to the cell surface by spin-inoculation. Additionally, we noticed that brazilin, TF-3, and curcumin can reduce fusion of spike-expressing cells to the hACE2 overdressing cellular monolayer. This confirmed our previous results indicating that all these three compounds have inhibitory properties directed especially towards RBD-SARS-CoV-2, and also suggest that they may also have an inhibitory effect on cellular proteases involved in SARS-CoV-2 infection steps. Our study did not elaborate on whether these polyphenols destroy viral particles, or whether they act by altering either the membranes of SARS-CoV-2 spike-enveloped pseudo-virions or the A549 cells. However, it has previously been shown that curcumin alters binding and fusion of the hepatitis C virus to the cell surface by affecting membrane fluidity.

Meanwhile, a study by Chen et al. documented that curcumin inhibits infectivity only of enveloped viruses, including the influenza virus. Since curcumin is a lipophilic molecule, it can induce the morphological changes in a membrane, reflected in disturbed integrity and increased fluidity, which may alter the conformation of both viral and host proteins. It has further been shown that theaflavins act as inhibitors of viral entry. For example, Chowdhury et al. found that theaflavins, including TF-3, inhibit the early steps of cellular entry of the hepatitis C virus, and suggested that they act directly on the viral particles rather than host cells blocking their dissemination. Cui et al. specifically reported on theaflavin-3,3′-digallate as an inhibitor of serine protease NS2B-NS3 of the Zika virus. Moreover, it was found from in silica study that theaflavins have a high binding affinity (i.e., ΔG of −8.53 kcal/mol) to the RBD of SARS-CoV-2 through forming hydrophobic interactions along with hydrogen bonds at ARG454, PHE456, ASN460, CYS480, GLN493, ASN501, and VAL503 of RBD-SARS-CoV-2, in proximity of the ACE2-spike protein contact area. Also, Maiti and Banerjee reported that theaflavin gallate prevents the RBD spike protein from binding to the hACE2 receptor.

Based on our results that corroborate the other published data, we cannot exclude that polyphenols tested in our study may also induce, directly or indirectly, allosteric interaction affecting other molecules and processes involved in SARS-CoV-2 infectivity. Thus, our further experiments were focused on molecules facilitating binding and entry of SARS-CoV-2, such as ACE2, TMPRSS2, and cathepsin L. Experiments in which the main attention was paid to ACE2 revealed that TF-3, and to a greater extent curcumin (but not brazilin), inhibit activity of ACE2 at non-toxic concentrations in both cell-free and cell-based assays. TF-3 and curcumin were shown to moderately bind to the hACE2 receptor at considerably low concentrations. Interestingly, none of these polyphenols down-regulated the expression of hACE2 at the protein level in A549 cells. This part of our study supports previously published computational prediction by Patel et al. and Jena et al. Also, Zhang et al., through docking screening, found that TF-3 could directly bind to the ACE2 receptor.

With regards to TMPRSS2, our experimental results showed that brazilin, TF-3, and curcumin can decrease activity of TMPRSS2 in cell-free and cell-based assays, but precisely how they inhibit its enzymatic activity, which reflects in interference with virus binding to the cell surface, remains to be established. Interestingly, as with hACE2, the protein expression level of TMPRSS2 was not affected. Our results further showed that Tf-3 and, again more profoundly, curcumin inhibit activity of cathepsin L in cell-free and cell-based assays. To add to this, all of the selected polyphenols, albeit to different extents, increased lysosomal/endosomal pH from around pH=5.0, concurring with previous reports, to around pH=6.0-6.5. This could have the effect on activity of cathepsin L. However, with regard to TF-3 and especially curcumin, in either direct or close proximity, binding could happen, since upon treatment with TF-3 and curcumin, inhibition of cathepsin L activity was statistically significant, though only mildly down-regulated upon treatment with brazilin. The precise mechanism for this inhibition, reflected in the interference with viral endosomal egress, could be further clarified by utilizing computational study.

Ravish et al. recognized curcumin as an inhibitor of cathepsin B and H, and found a correlation with results obtained from the computational docking experiment. In contrast to ACE2 and TMPRSS2 molecules, we observed also that TF-3, but not brazilin or curcumin, modestly decreased expression of cathepsin L at the protein level. Zhang et al. reported that curcumin increases the expression of cathepsin K and L in bleomycin-treated mice and human fibroblasts, while a study by Yoo et al. showed that expression of cathepsin L, elevated by palmitate in adipose tissue, can be inhibited by curcumin. This suggests that it is a tissue- and cell-specific process.

Altogether, our results show that brazilin, TF-3, and curcumin can affect critical mechanisms involved in SARS-CoV-2 cellular entry and internalization. This study also expands our knowledge of the number of viruses that are sensitive to curcumin and TF-3, and identifies novel polyphenol brazilin with anti-SARS-CoV-2 properties, highlighting the mechanism by which these polyphenols can act to inhibit SARS-CoV-2 infectivity. It remains to be investigated whether other cellular and viral molecules that contribute to SARS-CoV-2 infection could be affected by these polyphenols. Application of this class of compounds might unravel previously unidentified but important mechanisms to expand our understanding of SARS-CoV-2 biology. Particularly interesting would be details behind their efficacy in SARS-CoV-2 pathophysiology during later steps of the infection process. It also raises a question as to whether these polyphenols could be detrimental or beneficial for host responses following SARS-CoV-2 infection, and whether their antiviral potential could support or complement current pharmacological treatment.

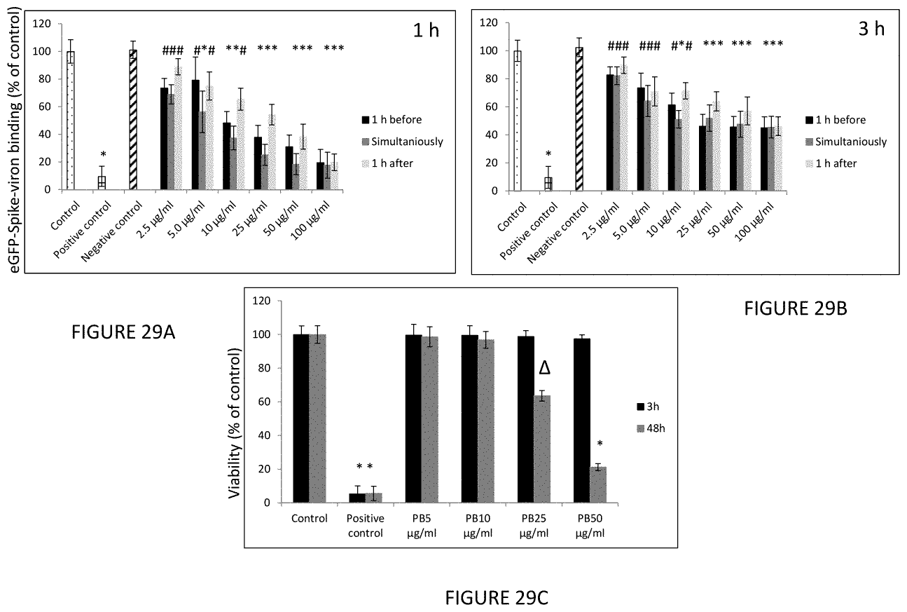

Effect of combination of polyphenols and plant extract (PB) on receptor binding: The effects of PB on attachment and entering of SARS-CoV-2 spike-enveloped virions were tested using lung cells stably overexpressing human ACE2 receptor (i.e., A549/hACE2 cells). The results presented in FIG. 29A, FIG. 29B, and FIG. 29C show the concentration-dependent inhibitory effects of PB on binding of the spike-encapsulated pseudo-virions to A549/hACE2 cells. PB was added to the pseudo-virions 1 hour before, simultaneously with the pseudo-virions, or 1 hour after A549/hACE2 cells were exposed to pseudo-virions. The resulting blockage of the virion binding was evaluated after 1 hour and 3 hours of exposure to the entire experimental mixture. The results show a concentration-dependent inhibition of viral binding to A549/hACE2 cells, with maximum inhibition obtained at 100 μg/ml PB concentration. At this concentration, similar levels of binding inhibition were observed in all three patterns of PB administration: 1 hour before, simultaneously, and 1 hour after virion-cells interaction, and after 1 hour and 3 hours of exposure of cells to virions together with PB.

The inhibitory effect on virion binding was more pronounced after 1 hour of exposure and equaled about 90% after factoring in positive control values (FIG. 29A). After 3 hours of exposure the maximum inhibitory effect achieved at PB concentration of 100 μg/ml was 55-60% (in relation to positive controls) and was basically similar for different PB exposure patterns (FIG. 29B). After 1 hour incubation period, PB at 10 μg/ml when added simultaneously with pseudo-virions and A549 cells, inhibited the binding by 63%, whereas 75% inhibition was observed when incubation time was extended to 3 hours. The inhibition obtained with a dose of 25 μg/ml after 1 hour and 3 hours was similar and equaled 51% and 52%, respectively, and observed at non-toxic concentrations (FIG. 29C).

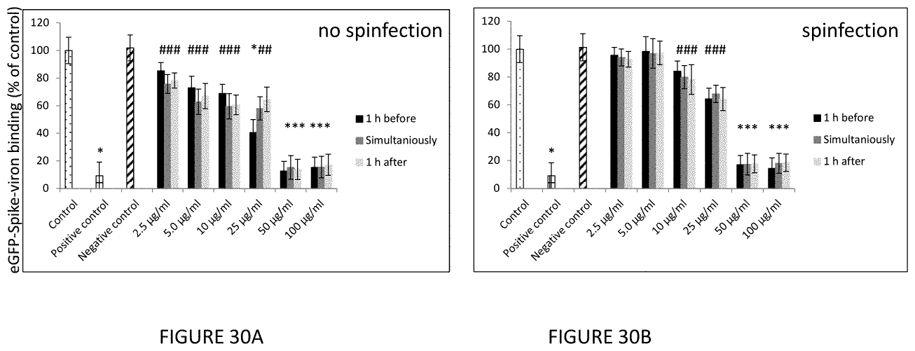

The effects of BP on the attachment and entry of pseudo-virions encapsulated with eGFP-luciferase spike protein were examined with and without spinfection in A549/hACE2 cells (FIGS. 30A and 30B). The results show that after 48 hours of incubation without spinfection there was a dose-dependent decrease in cell transduction by pseudo-virions by the PB. The differences in inhibitory effects between different application patterns were not statistically significant. Highest efficacy in binding inhibition was observed when virions were incubated for 1 hour with PB prior being added to the cells, compared with PB either simultaneous or 1-hour after addition with virions and cells. The results on FIG. 30B show that spinfection could facilitate the virions' binding, as the binding inhibition by corresponding PB concentrations was lower compared with non-infected cells. However, PB was still effective in causing about 20% binding inhibition at 10 μg/ml PB concentrations, respectively. PB beyond 25 μg/ml concentrations affected cell morphology that might contribute to the inhibitory effects as shown in FIG. 29C.

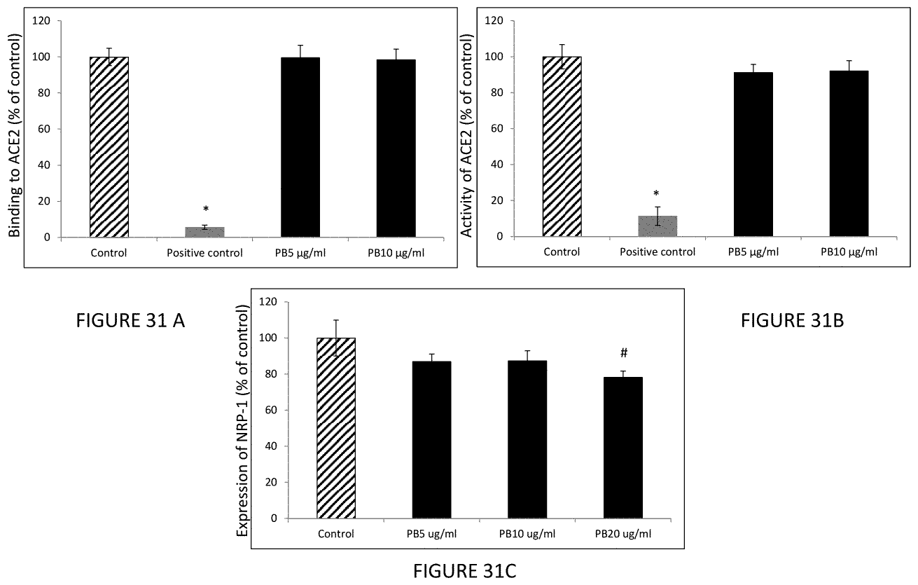

Effect of PB on host cellular receptors and proteases: It was already demonstrated that SARS-CoV-2 must attach to the ACE2 if it is to enter the host cell. Our previous results showed that PB interferes with attachment of the RBD of spike protein to the ACE2 molecule by directly binding to RBD sequence. The results in FIG. 31A show that PB did not bind to the ACE2 receptor or affect its activity as observed in free-cell assays. However, we observed dose-dependent down-regulation of cellular expression of NPR-1 (FIG. 31B), another receptor participating in SARS-CoV-2 cell entry and infectivity, showing the statistical significance at 20 μg/ml concentration (FIG. 31C).

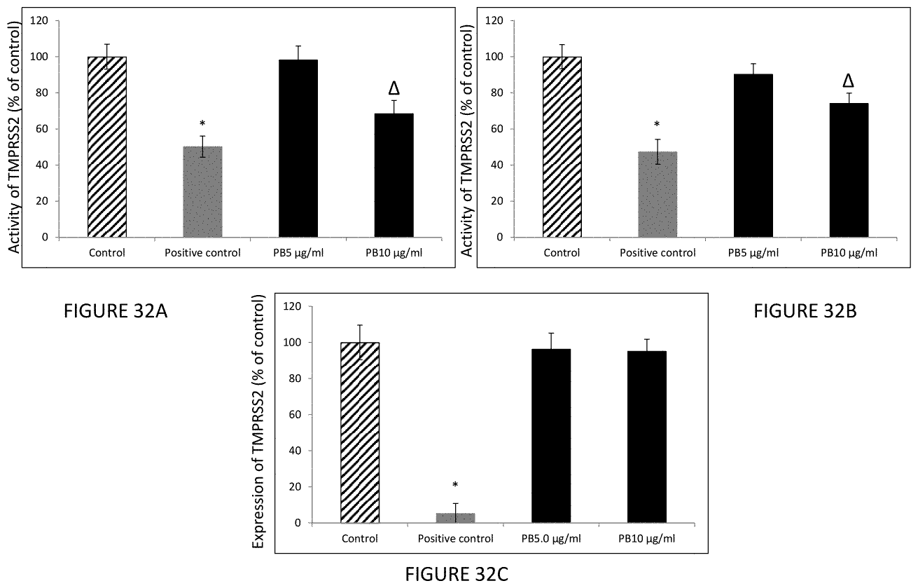

Except host receptors, specific cell surface proteases are also required to facilitate SARS-CoV-2 cellular entry by “priming” spike protein by enzymatic cleavage. These include TMPRSS2, cathepsin L, and furin, all implicated in viral binding and internalization. In our study we employed cell-free and cell-based assays to study the effects of PB on activity of these enzymes. As presented in FIGS. 32A and 32B, PB applied at 10 μg/ml showed statistically significant inhibition of TMPRSS2 activity in cell-free assay by about 31%. This enzyme activity assessed in A549 cells also resulted in a 25%© decrease in the presence of PB at 10 μg/ml concentration. This inhibition occurred in dose-dependent fashion and concurred with the concentrations that revealed to have inhibitory efficacy in viral binding. Interestingly, TPMRSS2 expression at protein level was not affected by PB at these concentrations (FIG. 32C).

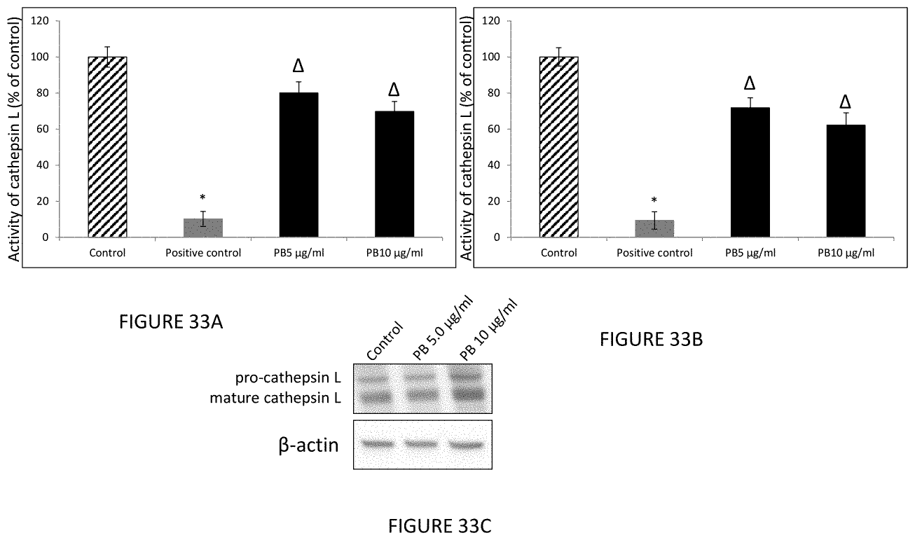

In addition, we tested the effects of PB on the activity of cathepsin L involved in SAIS-CoV-2 endosomal egress in both cell-free and cell-based assay. As shown in FIG. 33A the enzymatic activity of cathepsin L in cell-free assay was reduced by i′B in a dose-dependent fashion by 20% and by 30% at 5.0 and 10 μg/ml concentrations, respectively. Cathepsin L activity tested in A549 cells was lower by 22% and 37% upon treatment with 5.0 and 10 μg/ml concentrations, respectively (FIG. 33B). Cathepsin L expression at protein level was not affected by PB at these concentrations (FIG. 33C).

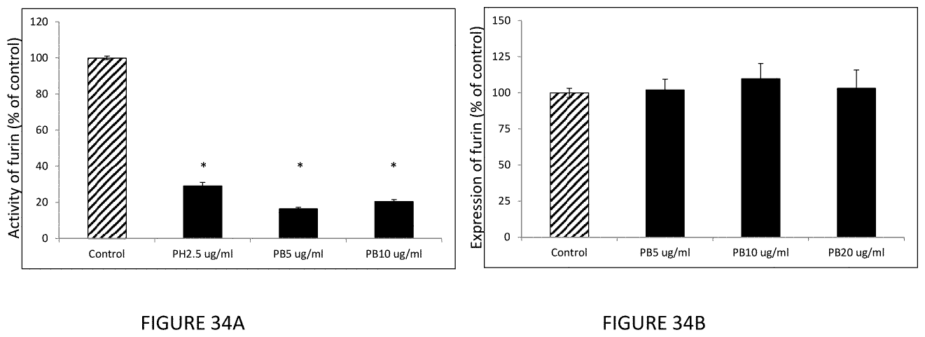

The effects of PB on furin activity and its cellular expression are presented in FIG. 34A and FIG. 34B. We observed concentration-dependent inhibition of the activity of furin in a cell-free assay with PB applied between 2.5 and 10 μg/ml. However, PB did not inhibit cellular expression of furin at non-toxic concentrations (i.e., up to 20 μg/ml).

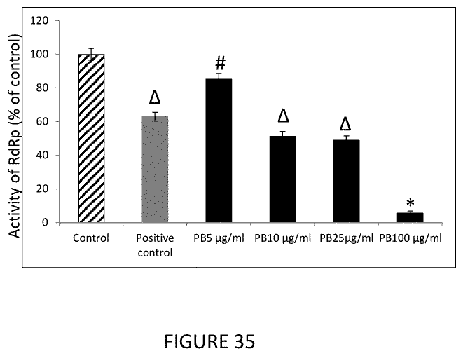

Effect of PB on viral RNA polymerase: In our study we also tested whether PB acts beyond the entry steps of the SARS-CoV-2 infection process, by examining whether PB at non toxic concentrations (i.e., up to 20 μg/ml) can inhibit the activity of recombinant RdRp. As presented in FIG. 35, PB's inhibitory effect on a SARS-CoV-2 RdRp was dose-dependent with ˜15% statistically significant inhibition achieved at 5.0 μg/ml and −49% at 10 μg/ml. Moreover, PB used at 100 μg/ml concentration inhibited RdRp activity by nearly 100%.

The results presented in this study show that a defined combination of active plant components and extracts (PB) can simultaneously inhibit key cellular steps involved in SARS-CoV-2 infection: its attachment to the ACE2 cellular receptor, and the activity of the key identified enzymes required for cellular entry and replication. These enzymes include TMPRSS2, furin, cathepsin L and RdRp. Our present findings complement our earlier study results with PB, which showed almost 90% inhibition of the expression of hACE2 on SAEC, thereby reducing the “entry points” for SARS-CoV-2 virus, and the inhibition of RBD sequence binding to ACE2 by 87%.

In our study we applied different experimental patterns in order to distinguish the PB effects on SARS-CoV-2 virion before it interacts with the cells, added simultaneously, and after the cells were exposed to the SARS-CoV-2 pseudo-virions. In short term study, we observed that inhibitory effect of PB on virion binding was similar when added at 100 μg/ml concentrations to the viral particles 1 hour before cell inoculations, simultaneously, and 1 hour after cell inoculation with the virions. However, at lower PB concentrations (i.e., up to 25 μg/ml), the highest and longer-lasting inhibition of viral particles binding to A549 cells was observed when virions were exposed to the PB for 1 hour before interacting with the cells. This would suggest direct interaction of the micronutrients with the viral particles, resulting in the inhibition of viral attachment to human cells. This observation was corroborated by the fact that we did not see any effects of PB on modulating ACE2 receptor binding properties and ACE2 enzymatic activity.

While PB had no effect on the activity of the ACE2, it merits particular attention in the light of the fact that PB significantly inhibits the cellular expression of ACE2 in SAEC. We interpret these observations as a function of a regulatory role of PB in cellular metabolism: while significantly reducing the expression of ACE2 receptors to a low physiological level, thereby limiting infectivity, PB does not affect the activity of these physiologically expressed ACE2 receptors. Such a regulatory effect of PB would be of particular significance, since ACE2 receptors have beneficial effects, e.g., in securing optimum cardiovascular function.

Most of the viruses use host enzymes for the proteolytic processing and maturation of their own proteins. It has been shown that, in addition to using the ACE2 receptor, SARS-CoV-2 virus implore NPR-1 molecule that shown dose-dependent cellular down-regulation upon treatment with PB as well as that its spike protein depends on proteolytic cleavage at the site between S1 and S2 and on S2 subunit to enable the fusion with and enter the target cell. Hence, the fusion capability of the CoV is a principal factor of their infection process. Among the proteolytic enzymes involved in the cleavage of spike protein, the TMPRSS2 activity has been shown to be vital for pathogenicity of SARS-CoV-2, accompanied by other enzymes such as cathepsins L. Furin is yet another protease involved in cleavage of mammalian, viral, and bacterial substrates. It has been shown that furin action towards the SARS-CoV spike protein is necessary for fusion of virions with host membranes without directly affecting viral infectivity. It appears that effective control and treatment of COVID-19 might necessitate parallel inhibition of several proteases to effectively obstruct these pathological conversions.

Here we have shown that, in addition to impairing viral binding to hACE2 overexpressing cells, the PB downregulated activity of key membrane proteases TMPRSS2, furin, and endosomal cathepsin L. In both cell-free and cell-based assays the reduction of the activity of TMPRSS2 and cathepsin L by PB was observed at its non-toxic concentrations. Furin activity, too, was significantly reduced at these relatively low PB concentrations. This effect is significant, as the lack of the additional furin cleavage site on the SARS-CoV spike protein has a substantial influence on its infectivity. In addition to SARS-CoV-2 infection, the potential signal link between spike protein, furin, and ACE2 has been implied in the occurrence of adverse cardiovascular events. Finally, we also recorded inhibited activity of RdRp at these concentrations, which would help to further explain a decreased transduction rate, even after applied spinfection.

Based on this study and our earlier findings, this combination of plant-derived compounds and plant extracts may constitute a new therapeutic strategy by simultaneously affecting viral entry, RdRp activity and ACE2 expression. Such a comprehensive effects of naturally occurring compounds on several mechanisms associated with viral infectivity is not surprising. This strategy was also implemented in our earlier studies, including those of human influenza H1N1, bird flu H1N5, and others, which were based on selecting natural components that simultaneously affect key pathology mechanisms across a wide spectrum of infective agents.

This study showed that definite combination of plant-derived, biologically active compounds can effectively in simultaneous manner control key steps of the SARS-CoV-2 infectivity.

Physiological dose levels for mammalian consumption were calculated based on various factors which include type of administration, species dependency and mode of action, such as transdermal vs oral. The range disclosed includes those factors along with scientific calculations. The range may differ within the range as well depending on formulations and species. Drug formulations suitable for these administration routes can be produced by adding one or more pharmacologically acceptable carrier to the agent and then treating the micronutrient composition through a routine process known to those skilled in the art. The mode of administration includes, but is not limited to, non-invasive peroral, topical (for example, transdermal), enteral, transmucosal, targeted delivery, sustained-release delivery, delayed release, pulsed release and parenteral methods. Peroral administration may be administered both in liquid and dry state.

Formulations suitable for oral administration may be in the form of capsules, cachets, pills, tablets, lozenges (using a flavored bases, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin or sucrose and acacia), each containing a predetermined amount of a subject composition as an active ingredient. Subject compositions may also be administered as a bolus, electuary or paste.

When an oral solid drug product is prepared, micronutrient composition is mixed with an excipient (and, if necessary, one or more additives such as a binder, a disintegrant, a lubricant, a coloring agent, a sweetening agent, and a flavoring agent), and the resultant mixture is processed through a routine method, to thereby produce an oral solid drug product such as tablets, coated tablets, granules, powder or capsules. Additives may be those generally employed in the art. Examples of excipients include lactate, sucrose, sodium chloride, glucose, starch, calcium carbonate, kaolin, microcrystalline cellulose and silicic acid. Binders include water, ethanol, propanol, simple syrup, glucose solution, starch solution, liquefied gelatin, carboxymethylcellulose, hydroxypropyl cellulose, hydroxypropyl starch, methyl cellulose, ethyl cellulose, shellac, calcium phosphate and polyvinyl pyrrolidone. Disintegrants include dried starch, sodium arginate, powdered agar, sodium hydroxy carbonate, calcium carbonate, sodium lauryl sulfate, monoglyceryl stearate and lactose. Lubricants include purified talc, stearic acid salts, borax and polyethylene glycol. Sweetening agents include sucrose, orange peel, citric acid and tartaric acid.

When a liquid drug product for oral administration is prepared, pharmaceutical micronutrient composition is mixed with an additive such as a sweetening agent, a buffer, a stabilizer, or a flavoring agent, and the resultant mixture is processed through a routine method, to produce an orally administered liquid drug product such as an internal solution medicine, syrup or elixir. Examples of the sweetening agent include vanillin; examples of the buffer include sodium citrate; and examples of the stabilizer include tragacanth, acacia, and gelatin.

For the purposes of transdermal (e.g., topical) administration, dilute sterile, aqueous or partially aqueous solutions (usually in about 0.1% to 5% concentration), otherwise similar to the above parenteral solutions, may be prepared with pharmaceutical micronutrient composition.

Formulations containing pharmaceutical micronutrient composition for rectal or vaginal administration may be presented as a suppository, which may be prepared by mixing a subject composition with one or more suitable non-irritating carriers, comprising, for example, cocoa butter, polyethylene glycol, a suppository wax or a salicylate, which is solid at room temperature, but liquid at body temperature and, therefore, will melt in the appropriate body cavity and release the encapsulated compound(s) and composition(s). Formulations that are suitable for vaginal administration also include pessaries, tampons, creams, gels, pastes, foams or spray formulations containing such carriers as are known in the art to be appropriate.

A targeted-release portion for capsules containing pharmaceutical micronutrient composition can be added to the extended-release system by means of either applying an immediate-release layer on top of the extended release core; using coating or compression processes, or in a multiple-unit system such as a capsule containing extended- and immediate-release beads.

When used with respect to a pharmaceutical micronutrient composition, the term “sustained release” is art recognized. For example, a therapeutic composition that releases a substance over time may exhibit sustained-release characteristics, in contrast to a bolus type administration in which the entire amount of the substance is made biologically available at one time. In particular embodiments, upon contact with body fluids, including blood, spinal fluid, mucus secretions, lymph or the like, one or more of the pharmaceutically acceptable excipients may undergo gradual or delayed degradation (e.g., through hydrolysis), with concomitant release of any material incorporated therein, e.g., a therapeutic and/or biologically active salt and/or composition, for a sustained or extended period (as compared with the release from a bolus). This release may result in prolonged delivery of therapeutically effective amounts of any of the therapeutic agents disclosed herein.

Current efforts in the area of drug delivery include the development of targeted delivery, in which the drug is only active in the target area of the body (for example, mucous membranes such as in the nasal cavity), and sustained-release formulations, in which the pharmaceutical micronutrient composition is released over a period of time in a controlled manner from a formulation. Types of sustained release formulations include liposomes, drug-loaded biodegradable microspheres and pharmaceutical micronutrient composition polymer conjugates.

Delayed-release dosage formulations are created by coating a solid dosage form with a film of a polymer, which is insoluble in the acid environment of the stomach, but soluble in the neutral environment of the small intestine. The delayed-release dosage units can be prepared, for example, by coating a pharmaceutical micronutrient composition with a selected coating material. The pharmaceutical micronutrient composition may be a tablet for incorporation into a capsule, a tablet for use as an inner core in a “coated core” dosage form, or a plurality of drug-containing beads, particles or granules, for incorporation into either a tablet or a capsule. Preferred coating materials include bioerodible, gradually hydrolysable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract, or slowly erode as the dosage form passes through the gastrointestinal tract, while enzymatically degradable polymers are degraded by bacterial enzymes present in the lower gastrointestinal tract, particularly in the colon. Alternatively, a delayed-release tablet may be formulated by dispersing a drug within a matrix of a suitable material such as a hydrophilic polymer or a fatty compound. Suitable hydrophilic polymers include, but are not limited to, polymers or copolymers of cellulose, cellulose ester, acrylic acid, methacrylic acid, methyl acrylate, ethyl acrylate and vinyl or enzymatically degradable polymers or copolymers as described above. These hydrophilic polymers are particularly useful for providing a delayed-release matrix. Fatty compounds for use as a matrix material include, but are not limited to, waxes (e.g., carnauba wax) and glycerol tristearate. Once the active ingredient is mixed with the matrix material, the mixture can be compressed into tablets.

A pulsed-release dosage is one that mimics a multiple dosing profile without repeated dosing, and typically allows at least a twofold reduction in dosing frequency as compared with the drug presented as a conventional dosage form (e.g., as a solution or prompt drug-releasing, conventional solid dosage form). A pulsed-release profile is characterized by a time period of no release (lag time) or reduced release, followed by rapid drug release. These can be formulated for critically ill patients using the instant pharmaceutical micronutrient composition.

The phrases “parenteral administration” and “administered parenterally” as used herein refer to modes of administration other than enteral and topical, such as injections, and include without limitation intravenous, intramuscular, intrapleural, intravascular, intrapericardial, intra-arterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

Certain pharmaceutical micronutrient composition disclosed herein, suitable for parenteral administration, comprise one or more subject compositions in combination with one or more pharmaceutically acceptable sterile, isotonic, aqueous, or non-aqueous solutions, dispersions, suspensions or emulsions, or sterile powders, which may be reconstituted into sterile injectable solutions or dispersions just prior to use, and which may contain antioxidants, buffers, bacteriostats, solutes that render the formulation isotonic within the blood of the intended recipient, or suspending or thickening agents.

When an injection product is prepared, pharmaceutical micronutrient composition is mixed with an additive such as a pH regulator, a buffer, a stabilizer, an isotonicity agent or a local anesthetic, and the resultant mixture is processed through a routine method, to thereby produce an injection for subcutaneous injection, intramuscular injection, or intravenous injection. Examples of the pH regulator or buffer include sodium citrate, sodium acetate and sodium phosphate; examples of the stabilizer include sodium pyrosulfite, EDTA, thioglycolic acid, and thiolactic acid; examples of the local anesthetic include procaine hydrochloride and lidocaine hydrochloride; and examples of the isotonicity agent include sodium chloride and glucose.

Adjuvants are used to enhance the immune response. Various types of adjuvants are available. Haptens and Freund's adjuvant may also be used to produce water-in-oil emulsions of immunogens.

The phrase “pharmaceutically acceptable” is art recognized. In certain embodiments, the term includes compositions, polymers and other materials and/or dosage forms that are within the scope of sound medical judgment, suitable for use in contact with the tissues of mammals, both human beings and animals, without excessive toxicity, irritation, allergic response or other problem or complication, commensurate with a reasonable benefit-risk ratio.

The phrase “pharmaceutically acceptable carrier” is art recognized, and includes, for example, pharmaceutically acceptable materials, compositions or vehicles, such as a liquid or solid filler, diluent, solvent or encapsulating material involved in carrying or transporting any subject composition from one organ or portion of the body, to another organ or portion of the body. Each carrier must be “acceptable” in the sense of being compatible with the other ingredients of a subject composition, and not injurious to the patient. In certain embodiments, a pharmaceutically acceptable carrier is non-pyrogenic. Some examples of materials that may serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the pharmaceutical micronutrient compositions described herein are formulated in a manner such that said compositions will be delivered to a mammal in a therapeutically effective amount, as part of a prophylactic, preventive or therapeutic treatment to overcome the infection caused by corona viruses (irrespective of the type).

In certain embodiments, the dosage of the pharmaceutical micronutrient compositions, which may be referred to as therapeutic composition provided herein, may be determined by reference to the plasma concentrations of the therapeutic composition or other encapsulated materials. For example, the blood samples may be tested for their immune response to their corresponding viral load or lack thereof.

The therapeutic pharmaceutical micronutrient composition provided by this application may be administered to a subject in need of treatment by a variety of conventional routes of administration, including orally, topically, parenterally, e.g., intravenously, subcutaneously or intramedullary. Further, the therapeutic compositions may be administered intranasally, as a rectal suppository, or using a “flash” formulation, i.e., allowing the medication to dissolve in the mouth without the need to use water. Furthermore, the compositions may be administered to a subject in need of treatment by controlled-release dosage forms, site-specific drug delivery, transdermal drug delivery, patch-mediated drug delivery (active/passive), by stereotactic injection, or in nanoparticles.

Expressed in terms of concentration, an active ingredient can be present in the therapeutic micronutrient compositions of the present invention for localized use via the cutis, intranasally, pharyngolaryngeally, bronchially, intravaginally, rectally or ocularly.

For use as aerosols, the active ingredients can be packaged in a pressurized aerosol container together with a gaseous or liquefied propellant, for example dichlorodifluoromethane, carbon dioxide, nitrogen, propane and the like, with the usual adjuvants such as cosolvents and wetting agents, as may be necessary or desirable. The most common routes of administration also include the preferred transmucosal (nasal, buccal/sublingual, vaginal, ocular and rectal) and inhalation routes.

In addition, in certain embodiments, the subject micronutrient composition of the present application may be lyophilized or subjected to another appropriate drying technique such as spray drying. The subject micronutrient compositions may be administered once, or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject pharmaceutical micronutrient composition that may be combined with a carrier material to produce a single dose may vary depending upon the subject being treated and the particular mode of administration.

The therapeutically acceptable amount described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may, for example, contain 0.005-90% w/w, for instance 0.005-50%, 0.005-5% w/w, or 0.01-1.0% w/w, of medicament relative to the total weight of the formulation.

Examples of suitable aqueous and non-aqueous carriers that may be employed in the pharmaceutical micronutrient composition include water, ethanol, polyols (such as glycerol, propylene glycol, polyethylene glycol and the like), and suitable mixtures thereof, vegetable oils such as olive oil, and injectable organic esters such as ethyl oleate. Proper fluidity may be maintained, for example by the use of coating materials such as lecithin, by the maintenance of the required particle size in the case of dispersions, and by the use of surfactants.

In conclusion, this study demonstrates pleiotropic anti-SARS-CoV-2 efficacy of specific polyphenols. This study indicates that a natural formulation of plant-derived active compounds can be effective in inhibiting the viral binding to ACE2 receptors and at the same time it can significantly decrease cellular expression of ACE2 receptors on lung alveolar epithelial cells. This natural approach shows that key mechanisms involved in viral infectivity can be controlled simultaneously, increasing a chance of success and may constitute a new therapeutic strategy to deal with this unprecedented and powerful virus threat.

The present invention preferably relates to the following two sets of items: A micronutrient composition to prevent or treat a SARS-CoV-2 virus infection by inhibiting attachment to a cognate receptor, cellular entry, replication and cellular egress of the SARS-CoV-2 virus in a mammal, comprising; a phenolic acid, plant extracts, flavonoid, stilbenes, alkaloid, terpene, vitamin, volatile oil, mineral, fatty acids polyunsaturated, fatty acids monounsaturated individually or in combination thereof, wherein the phenolic acid are at least one of a tannic acid, (+) epigallocatechin gallate, (−)-gallocatechin gallate, curcumin and a combination thereof, wherein plant extracts are at least one of a quercetin, cruciferous extract, turmeric root extract, green tea extract and resveratrol, wherein flavonoid is at least one of a hesperidin, brazilin, phloroglucinol and myricetin, wherein alkaloid is at least one of a palmatine and usnic acid, wherein terpene is at least one of a D-limonene and carnosic acid, wherein stilbenes is a trans-resveratrol, wherein the vitamin is at least one of a vitamin C, vitamin E, vitamin B1, vitamin B2, vitamin B3, vitamin B6, vitamin B12, folate, biotin and a combination thereof, wherein the volatile oils are at least one of a eugenol oil from clove oil, oregano oil, carvacrol, cinnamon oil, thyme oil, trans-trans-cinnamaldehyde, wherein fatty acid polyunsaturated are at least one of a linolenic acid, eicosapentaenoic acid, docosahexaenoic acid, linoleic acid, wherein fatty acid monounsaturated are at least one of a oleic acid, baicalin, luteolin, hesperidin, tea extract, medium chain triglycerides, Skullcap root extract, Rosemary leaf extract, Royal Jelly, selenium, copper, manganese, iodine (kelp), L-lysine, L-arginine, L-proline, N-acetylcysteine, alpha lipoic acid and petroselinic acid and any combination thereof. The second being: The micronutrient composition of item 1, wherein the micronutrients as a composition are present in in between a range of: the tannic acid 1 mg-200 mg, (+) epigallocatechin gallate 1 mg-5000 mg, (−)-gallocatechin gallate 1 mg-5000 mg, curcumin 1 mg-10000 mg, quercetin 1 mg-2000 mg, cruciferous extract 1 mg-5000 mg, turmeric root extract 1 mg-30000 mg, green tea extract 1 mg-20000 mg, resveratrol 1 mg-50000 mg, hesperidin 1 mg-2000 mg, brazilin 1 mg-1000 mg, phloroglucinol 1 mg-100 mg, myricetin 1 mg-1000 mg, wherein alkaloid is at least one of a palmatine and usnic acid, D-limonene 1 mg-1500 mg, carnosic acid 1 mg 700 mg, trans-resveratrol 1 mg-3,000 mg, vitamin C 10 mg-100000 mg, vitamin E 1 mg-3,000 mg, vitamin B1 1 mg-3000 mg, vitamin B2 1 mg-2000 mg, vitamin B3 1 mg-3000 mg, vitamin B6 1 mg-3000 mg, vitamin B12 10 mcg-2000 mcg, folate 1 mcg-3000 mcg, biotin 1 mg-20000 mg, eugenol oil from clove oil 1 mg-300 mg, oregano oil 1 mg-1000 mg, carvacrol 1 mg-500 mg, cinnamon oil 1 mg-1000 mg, thyme oil 0.1 mg-100 mg, trans-trans-cinnamaldehyde 1 mg-4000 mg, linolenic acid 1 mg-8000 mg, eicosapentaenoic acid 1 mg-8000 mg, docosahexaenoic acid 1 mg-8000 mg, linoleic acid 1 mg-8000 mg, oleic acid 1 mg-20000 mg and petroselinic acid 1 mg-4000 mg, baicalin 1 mg-4000 mg, luteolin 0.1 mg-100 mg, hesperidin 1 mg-2000 mg, tea extract 0.1 mg-10000 mg, medium chain triglycerides 1 mg-70000 mg, Skullcap root extract 1 mg-5000 mg, Rosemary leaf extract 1 mg-10000 mg, Royal Jelly 1 mg-10000 mg, selenium 2 mcg-500 mcg, copper 0.01 mg-20 mg, manganese 1 mg-30 mg, iodine (kelp) 0.01 mg-2 mg, L-lysine 1 mg-40000 mg, L-arginine 1 mg-30000 mg, L-proline 1 mg-20000 mg, N-acetylcysteine 1 mg-30000 mg, alpha lipoic acid 1 mg-5,000 mg and any combination thereof. In one embodiment, the micronutrient composition of item 2, consists of the quercetin 1 mg-2000 mg, cruciferous extract 1 mg-5000 mg, turmeric root extract 1 mg-30000 mg, green tea extract 1 mg-20000 mg, resveratrol 1 mg-50000 mg, baicalin 1 mg-4000 mg, luteolin 0.1 mg-100 mg, hesperidin 1 mg-2000 mg, tea extract 0.1 mg-10000 mg and a combination thereof. Still another embodiment, the micronutrient composition of item 2, consists of the quercetin 1 mg-2000 mg, cruciferous extract 1 mg-5000 mg, turmeric root extract 1 mg-30000 mg, green tea extract 1 mg-20000 mg, resveratrol 1 mg-50000 mg and a combination thereof. In another embodiment, the micronutrient composition of item 2, consisting of the vitamin C 10 mg-100000 mg, selenium 2 mcg-500 mcg, copper 0.01 mg-20 mg, manganese 1 mg-30 mg, L-lysine 1 mg-40000 mg, L-arginine 1 mg-30000 mg, L-proline 1 mg-20000 mg, N-acetylcysteine 1 mg-30000 mg, quercetin 1 mg-2000 mg, green tea extract 1 mg-20000 mg and a combination thereof. Another additional combination of the micronutrient composition of item 2, consists of the iodine (kelp) 0.01 mg-2 mg, luteolin 0.1 mg-100 mg, medium chain triglycerides 1 mg-70000 mg, Skullcap root extract 1 mg-5000 mg, Rosemary leaf extract 1 mg-10000 mg, Royal Jelly 1 mg-10000 mg and a combination thereof.

In one embodiment, a micronutrient composition, comprises of a tannic acid 1 mg-200 mg, (+) epigallocatechin gallate 1 mg-5000 mg, (−)-gallo catechin gallate 1 mg-5000 mg, curcumin 1 mg-10000 mg, quercetin 1 mg-2000 mg, cruciferous extract 1 mg-5000 mg, turmeric root extract 1 mg-30000 mg, green tea extract 1 mg-20000 mg, resveratrol 1 mg-50000 mg, hesperidin 1 mg-2000 mg, brazilin 1 mg-1000 mg, phloroglucinol 1 mg-100 mg, myricetin 1 mg-1000 mg, wherein alkaloid is at least one of a palmatine and usnic acid, D-limonene 1 mg-1500 mg, carnosic acid 1 mg-700 mg, trans-resveratrol 1 mg-3,000 mg, vitamin C 10 mg-100000 mg, vitamin E 1 mg-3,000 mg, vitamin B1 1 mg-3000 mg, vitamin B2 1 mg-2000 mg, vitamin B3 1 mg-3000 mg, vitamin B6 1 mg-3000 mg, vitamin B12 10 mcg-2000 mcg, folate 1 mcg-3000 mcg, biotin 1 mg-20000 mg, eugenol oil from clove oil 1 mg-300 mg, oregano oil 1 mg-1000 mg, carvacrol 1 mg-500 mg, cinnamon oil 1 mg-1000 mg, thyme oil 0.1 mg-100 mg, trans-trans-cinnamaldehyde 1 mg-4000 mg, linolenic acid 1 mg-8000 mg, eicosapentaenoic acid 1 mg-8000 mg, docosahexaenoic acid 1 mg-8000 mg, linoleic acid 1 mg-8000 mg, oleic acid 1 mg-20000 mg, petroselinic acid 1 mg-4000 mg, baicalin 1 mg-4000 mg, luteolin 0.1 mg-100 mg, hesperidin 1 mg-2000 mg, tea extract 0.1 mg-10000 mg, medium chain triglycerides 1 mg-70,000 mg, Skullcap root extract 1 mg-5,000 mg, Rosemary leaf extract 1 mg-10,000 mg, Royal Jelly 1 mg-10,000 mg, selenium 2 mcg-500 mcg, copper 0.01 mg-20 mg, manganese 1 mg-30 mg, iodine (kelp) 0.01 mg-2 mg, L-lysine 1 mg-40,000 mg, L-arginine 1 mg-30,000 mg, L-proline 1 mg-20,000 mg, N-acetylcysteine 1 mg-30,000 mg, alpha lipoic acid 1 mg-5,000 mg and any combination thereof to inhibit attachment to a cognate receptor, cellular entry, replication and cellular egress of a SARS-CoV-2 virus in a mammal.

The micronutrient composition stated above further comprises of the quercetin 1 mg-2000 mg, cruciferous extract 1 mg-5000 mg, turmeric root extract 1 mg-30000 mg, green tea extract 1 mg-20000 mg, resveratrol 1 mg-50000 mg, baicalin 1 mg-4000 mg, luteolin 0.1 mg-100 mg, hesperidin 1 mg-2000 mg, tea extract 0.1 mg-10000 mg and a combination thereof in another embodiment. In yet another embodiment. The micronutrient composition above, further consists of the quercetin 1 mg-2000 mg, cruciferous extract 1 mg-5000 mg, turmeric root extract 1 mg-30000 mg, green tea extract 1 mg-20000 mg, resveratrol 1 mg-50000 mg and a combination thereof. Yet another embodiment consists of the vitamin C 10 mg-100000 mg, selenium 2 mcg-500 mcg, copper 0.01 mg-20 mg, manganese 1 mg-30 mg, L-lysine 1 mg-40000 mg, L-arginine 1 mg-30000 mg, L-proline 1 mg-20000 mg, N-acetylcysteine 1 mg-30000 mg, quercetin 1 mg-2000 mg, green tea extract 1 mg-20000 mg and a combination thereof. The combination of the micronutrient composition further consists of the iodine (kelp) 0.01 mg-2 mg, luteolin 0.1 mg-100 mg, medium chain triglycerides 1 mg-70000 mg, Skullcap root extract 1 mg-5000 mg, Rosemary leaf extract 1 mg-10000 mg, Royal Jelly 1 mg-10000 mg and a combination thereof.

According to a further embodiment of the present invention, the present invention is defined by the following items: a composition comprising a flavonol, flavanol, a curcuminoid, and a phytoalexin, wherein the flavonol is quercetin, the flavanol is a catechin compound, preferably epigallocatechin gallate, and the phytoalexin is resveratrol. The composition above further contains cruciferous extract, broccoli extract or a mixture thereof, preferably in a daily dosage amount of 40 to 1200 mg. The composition further contains ascorbic acid or ascorbate, and caffeine. In the compositions above the flavanol is green tea extract. The composition of one of items 1 to 5, further containing one or more of baicalin, luteolin, hesperidin, theaflavin compounds. The composition of one of items 1 to 6, containing a daily dosage amount of 50 to 1600 mg flavonol, 40 to 1200 mg curcuminoid, 40 to 1200 mg flavanol, and 10 to 200 mg phytoalexin. A pharmaceutical composition comprising a composition according to one of items declared above and, if necessary, a pharmaceutically acceptable carrier. The pharmaceutical composition of item 8 for preventing and treating a. Covid-19 infection.

The pharmaceutical composition is for preventing, treating and delaying attachment, penetration, biosynthesis, maturation and release of a SARS-Cov-2 virus in a mammal or human. The pharmaceutical composition stated above is used for inhibiting the viral RBD binding to ACE2 receptors. The pharmaceutical composition as stated above, comprises one or more polyphenols, plant extracts, volatile oils, polyunsaturated fatty acids, monounsaturated fatty acids and lipid soluble vitamins. The flavonol is preferably a flavonoid and more preferably quercetin. This group of compounds belongs to polyphenols. The flavanol is preferably a flavan-3-ol or catechin compound, preferably epigallocatechin gallate. It belongs to the group of flavanoids and is preferably employed in the form of tea extract, more preferably green tea extract (EGCG). A preferred green tea extract contains the flavonoids, preferably black tea extract as flavonoid, caffeine and ascorbic acid or ascorbate (vitamin C). The phytoalexin is a polyphenol and is preferably resveratrol further component of the composition which is preferably included in the composition is a cruciferous extract, broccoli extract or a mixture thereof. Broccoli extract can also be regarded as a specific form of cruciferous extract. The curcuminoid is preferably curcumin which is part of the root extract of Curcuma longa, also known as turmeric root extract.

1. A composition comprising a flavonol, flavanol, a curcuminoid, and a phytoalexin, wherein the flavonol is quercetin, the phytoalexin is resveratrol, the flavanol is green tea extract, further containing calciferous extract, broccoli extract or a mixture thereof.

2. The composition of claim 1, further containing ascorbic acid or ascorbate, and caffeine.

3. The composition of one of claim 1 or 2, further containing one or more of baicalin, luteolin, hesperidin, theaflavin compounds.

4. The composition of one of claims 1 to 3, containing a daily dosage amount of 50 to 1600 mg flavonol, 40 to 1200 mg curcuminoid, 40 to 1200 mg flavanol, and 10 to 200 mg phytoalexin.

5. A pharmaceutical composition comprising a composition according to one of claims 1 to 4 and, if necessary, a pharmaceutically acceptable carrier.

6. The pharmaceutical composition of claim 5 for preventing and treating a Covid-19 infection.

7. The pharmaceutical composition of claim 5 or 6 for preventing, treating and delaying attachment, penetration, biosynthesis, maturation and release of a SARS-Cov-2 virus in a mammal or human.

8. The pharmaceutical composition of one of claims 5 to 7 for inhibiting the viral RBD binding to ACE2 receptors.

9. The pharmaceutical composition of one of claims 5 to 8, comprising one or more polyphenols, plant extracts, volatile oils, polyunsaturated fatty acids, monounsaturated fatty acids and lipid soluble vitamins.

10. A micronutrient composition comprising;
a phenolic acid, plant extracts, flavonoid, stilbenes, alkaloid, terpene, vitamin, volatile oil, mineral, polyunsaturated fatty acids, monounsaturated fatty acids, or in addition or as alternative to monounsaturated fatty acids one or both of medium chain triglycerides and alpha lipoic acid, in combination thereof, wherein the phenolic acid are at least one of a tannic acid, (+) epigallocatechin gallate, (−)-gallocatechin gallate, curcumin, wherein plant extracts are at least one of cruciferous extract, turmeric root extract, green tea extract, wherein flavonoid is at least one of a hesperidin, brazilin, phloroglucinol and myricetin, wherein alkaloid is at least one of a palmatine and usnic acid, wherein terpene is at least one of a D-limonene and carnosic acid, wherein stilbenes is a trans-resveratrol, wherein the vitamin is at least one of a vitamin C, vitamin E, vitamin B1, vitamin B2, vitamin B3, vitamin B6, vitamin B12, folate, biotin, wherein the volatile oils are at least one of a eugenol oil from clove oil, oregano oil, carvacrol, cinnamon oil, thyme oil, trans-trans-cinnamaldehyde, wherein polyunsaturated fatty acid are at least one of a linolenic acid, eicosapentaenoic acid, docosahexaenoic acid, linoleic acid, wherein monounsaturated fatty acid are at least one of a oleic acid, and petroselinic acid, wherein, the micronutrient composition comprises; the quercetin 1 mg-2000 mg, cruciferous extract 1 mg-5000 mg, turmeric root extract 1 mg-30000 mg, green tea extract 1 mg-20000 mg, resveratrol 1 mg-50000 mg.

11. The micronutrient composition of claim 10, wherein the micronutrients as a composition are present in in between a range of:
the tannic acid 1 mg-200 mg, (+) epigallocatechin gallate 1 mg-5000 mg, (−)-gallocatechin gallate 1 mg-5000 mg, curcumin 1 mg-10000 mg, quercetin 1 mg-2000 mg, cruciferous extract 1 mg-5000 mg, turmeric root extract 1 mg-30000 mg, green tea extract 1 mg-20000 mg, resveratrol 1 mg-50000 mg, hesperidin 1 mg-2000 mg, brazilin 1 mg-1000 mg, phloroglucinol 1 mg-100 mg, myricetin 1 mg-1000 mg, wherein alkaloid is at least one of a palmatine and usnic acid, D-limonene 1 mg-1500 mg, carnosic acid 1 mg 700 mg, trans-resveratrol 1 mg-3,000 mg, vitamin C 10 mg-100000 mg, vitamin E 1 mg-3,000 mg, vitamin B1 1 mg-3000 mg, vitamin B2 1 mg-2000 mg, vitamin B3 1 mg-3000 mg, vitamin B6 1 mg-3000 mg, vitamin B12 10 mcg-2000 mcg, folate 1 mcg-3000 mcg, biotin 1 mg-20000 mg, eugenol oil from clove oil 1 mg-300 mg, oregano oil 1 mg-1000 mg, carvacrol 1 mg-500 mg, cinnamon oil 1 mg-1000 mg, thyme oil 0.1 mg-100 mg, trans-trans-cinnamaldehyde 1 mg-4000 mg, linolenic acid 1 mg-8000 mg, eicosapentaenoic acid 1 mg-8000 mg, docosahexaenoic acid 1 mg-8000 mg, linoleic acid 1 mg-8000 mg, oleic acid 1 mg-20000 mg and petroselinic acid 1 mg-4000 mg, baicalin 1 mg-4000 mg, luteolin 0.1 mg-100 mg, hesperidin 1 mg-2000 mg, tea extract 0.1 mg-10000 mg, medium chain triglycerides 1 mg-70000 mg, Skullcap root extract 1 mg-5000 mg, Rosemary leaf extract 1 mg-10000 mg, Royal Jelly 1 mg-10000 mg, selenium 2 mcg-500 mcg, copper 0.01 mg-20 mg, manganese 1 mg-30 mg, iodine (kelp) 0.01 mg-2 mg, L-lysine 1 mg-40000 mg, L-arginine 1 mg-30000 mg, L-proline 1 mg-20000 mg, N-acetylcysteine 1 mg-30000 mg, alpha lipoic acid 1 mg-5,000 mg.

12. The micronutrient composition of claim 10, comprising;
the quercetin 1 mg-2000 mg, cruciferous extract 1 mg-5000 mg, turmeric root extract 1 mg-30000 mg, green tea extract 1 mg-20000 mg, resveratrol 1 mg-50000 mg, baicalin 1 mg-4000 mg, luteolin 0.1 mg-100 mg, hesperidin 1 mg-2000 mg, tea extract 0.1 mg-10000 mg.

13. The micronutrient composition of claim 10, comprising
the vitamin C 10 mg-100000 mg, selenium 2 mcg-500 mcg, copper 0.01 mg-20 mg, manganese 1 mg-30 mg, L-lysine 1 mg-40000 mg, L-arginine 1 mg-30000 mg, L-proline 1 mg-20000 mg, N-acetylcysteine 1 mg-30000 mg, quercetin 1 mg-2000 mg, green tea extract 1 mg-20000 mg.

14. The micronutrient composition of claim 10, comprising
the iodine (kelp) 0.01 mg-2 mg, luteolin 0.1 mg-100 mg, medium chain triglycerides 1 mg-70000 mg, Skullcap root extract 1 mg-5000 mg, Rosemary leaf extract 1 mg-10000 mg, Royal Jelly 1 mg-10000 mg.

15. A micronutrient composition, comprising or consisting of:
a tannic acid 1 mg-200 mg, (+) epigallocatechin gallate 1 mg-5000 mg, (−)-gallo catechin gallate 1 mg-5000 mg, curcumin 1 mg-10000 mg, quercetin 1 mg-2000 mg, cruciferous extract 1 mg-5000 mg, turmeric root extract 1 mg-30000 mg, green tea extract 1 mg-20000 mg, resveratrol 1 mg-50000 mg, hesperidin 1 mg-2000 mg, brazilin 1 mg-1000 mg, phloroglucinol 1 mg-100 mg, myricetin 1 mg-1000 mg, wherein alkaloid is at least one of a palmatine and usnic acid, D-limonene 1 mg-1500 mg, carnosic acid 1 mg-700 mg, trans-resveratrol 1 mg-3,000 mg, vitamin C 10 mg-100000 mg, vitamin E 1 mg-3,000 mg, vitamin B1 1 mg-3000 mg, vitamin B2 1 mg-2000 mg, vitamin B3 1 mg-3000 mg, vitamin B6 1 mg-3000 mg, vitamin B12 10 mcg-2000 mcg, folate 1 mcg-3000 mcg, biotin 1 mg-20000 mg, eugenol oil from clove oil 1 mg-300 mg, oregano oil 1 mg-1000 mg, carvacrol 1 mg-500 mg, cinnamon oil 1 mg-1000 mg, thyme oil 0.1 mg-100 mg, trans-trans-cinnamaldehyde 1 mg-4000 mg, linolenic acid 1 mg-8000 mg, eicosapentaenoic acid 1 mg-8000 mg, docosahexaenoic acid 1 mg-8000 mg, linoleic acid 1 mg-8000 mg, oleic acid 1 mg-20000 mg, petroselinic acid 1 mg-4000 mg, baicalin 1 mg-4000 mg, luteolin 0.1 mg-100 mg, hesperidin 1 mg-2000 mg, tea extract 0.1 mg-10000 mg, medium chain triglycerides 1 mg-70,000 mg, Skullcap root extract 1 mg-5,000 mg, Rosemary leaf extract 1 mg-10,000 mg, Royal Jelly 1 mg-10,000 mg, selenium 2 mcg-500 mcg, copper 0.01 mg-20 mg, manganese 1 mg-30 mg, iodine (kelp) 0.01 mg-2 mg, L-lysine 1 mg-40,000 mg, L-arginine 1 mg-30,000 mg, L-proline 1 mg-20,000 mg, N-acetylcysteine 1 mg-30,000 mg, alpha lipoic acid 1 mg-5,000 mg to inhibit attachment to a cognate receptor, cellular entry, replication and cellular egress of a SARS-CoV-2 virus in a mammal, wherein the micronutrient composition comprises; the quercetin 1 mg-2000 mg, cruciferous extract 1 mg-5000 mg, turmeric root extract 1 mg-30000 mg, green tea extract 1 mg-20000 mg, resveratrol 1 mg-50000 mg.

16. The micronutrient composition of claim 15, comprising;
the quercetin 1 mg-2000 mg, cruciferous extract 1 mg-5000 mg, turmeric root extract 1 mg-30000 mg, green tea extract 1 mg-20000 mg, resveratrol 1 mg-50000 mg, baicalin 1 mg-4000 mg, luteolin 0.1 mg-100 mg, hesperidin 1 mg-2000 mg, tea extract 0.1 mg-10000 mg.

17. The micronutrient composition of claim 15, comprising
the vitamin C 10 mg-100000 mg, selenium 2 mcg-500 mcg, copper 0.01 mg-20 mg, manganese 1 mg-30 mg, L-lysine 1 mg-40000 mg, L-arginine 1 mg-30000 mg, L-proline 1 mg-20000 mg, N-acetylcysteine 1 mg-30000 mg, quercetin 1 mg-2000 mg, green tea extract 1 mg-20000 mg.

18. The micronutrient composition of claim 15, comprising
the iodine (kelp) 0.01 mg-2 mg, luteolin 0.1 mg-100 mg, medium chain triglycerides 1 mg-70000 mg, Skullcap root extract 1 mg-5000 mg, Rosemary leaf extract 1 mg-10000 mg, Royal Jelly 1 mg-10000 mg.

19. The use of a micronutrient composition as claimed in one of claims 10 to 18 to prevent or to treat a SARS-CoV-2 virus infection by inhibiting attachment to a cognate receptor, cellular entry, replication and cellular egress of the SARS-CoV-2 virus in a mammal.

20. A pharmaceutical composition, preferably to prevent or to treat a SARS-CoV-2 virus infection by inhibiting attachment to a cognate receptor, cellular entry, replication and cellular egress of the SARS-CoV-2 virus in a mammal, comprising a micronutrient composition as claimed in one of claims 10 to 18 and optionally a pharmaceutically acceptable carrier.

21. The pharmaceutical composition as claimed in claim 20 in a form that allows for oral, parental, drip-feeding or gavage administration.
